SEQUENCE LISTING (1 sequence)

SEQ ID NO: 1 - RNA, 1653 nt, Artificial Sequence
  synthetic construct
  misc_feature (1)..(1653): Cytidine residues of the entire sequence may be unmodified
  misc_feature (1)..(1653): Cytidine residues of the entire sequence may be N4-methylcytidine
  misc_feature (1)..(1653): Cytidine residues of the entire sequence may be N4, 2′-O-dimethylcytidine
auggaggacgccaagaacaucaagaagggccccgcccccuucuacccccuggaggacggcaccgccggcgagcagcugcacaaggccaugaagcgguacgcccuggugcccggcaccaucgccuucaccgacgcccacaucgagguggacaucaccuacgccgaguacuucgagaugagcgugcggcuggccgaggccaugaagcgguacggccugaacaccaaccaccggaucguggugugcagcgagaacagccugcaguucuucaugcccgugcugggcgcccuguucaucggcguggccguggcccccgccaacgacaucuacaacgagcgggagcugcugaacagcaugggcaucagccagcccaccgugguguucgugagcaagaagggccugcagaagauccugaacgugcagaagaagcugcccaucauccagaagaucaucaucauggacagcaagaccgacuaccagggcuuccagagcauguacaccuucgugaccagccaccugccccccggcuucaacgaguacgacuucgugcccgagagcuucgaccgggacaagaccaucgcccugaucaugaacagcagcggcagcaccggccugcccaagggcguggcccugccccaccggaccgccugcgugcgguucagccacgcccgggaccccaucuucggcaaccagaucauccccgacaccgccauccugagcguggugcccuuccaccacggcuucggcauguucaccacccugggcuaccugaucugcggcuuccggguggugcugauguaccgguucgaggaggagcuguuccugcggagccugcaggacuacaagauccagagcgcccugcuggugcccacccuguucagcuucuucgccaagagcacccugaucgacaaguacgaccugagcaaccugcacgagaucgccagcggcggcgccccccugagcaaggaggugggcgaggccguggccaagcgguuccaccugcccggcauccggcagggcuacggccugaccgagaccaccagcgccauccugaucacccccgagggcgacgacaagcccggcgccgugggcaagguggugcccuucuucgaggccaaggugguggaccuggacaccggcaagacccugggcgugaaccagcggggcgagcugugcgugcggggccccaugaucaugagcggcuacgugaacaaccccgaggccaccaacgcccugaucgacaaggacggcuggcugcacagcggcgacaucgccuacugggacgaggacgagcacuucuucaucguggaccggcugaagagccugaucaaguacaagggcuaccagguggcccccgccgagcuggagagcauccugcugcagcaccccaacaucuucgacgccggcguggccggccugcccgacgacgacgccggcgagcugcccgccgccgugguggugcuggagcacggcaagaccaugaccgagaaggagaucguggacuacguggccagccaggugaccaccgccaagaagcugcggggcggcgugguguucguggacgaggugcccaagggccugaccggcaagcuggacgcccggaagauccgggagauccugaucaaggccaagaagggcggcaagaucgccguguga

(19) United States
(12) Patent Application Publication
(10) Pub. No.: US 2023/0219994 A1
(43) Pub. Date: Jul. 13, 2023

(54) MODIFIED NUCLEOSIDE AND SYNTHESIS METHOD THEREFOR

(71) Applicant: STEMIRNA THERAPEUTICS CO., LTD., Shanghai (CN)

(72) Inventors: Yunsong CAI, Shanghai (CN); Hangwen Li, Shanghai (CN); Lianxiao Liu, Shanghai (CN); Ning Li, Shanghai (CN)

(21) Appl. No.: 17/635,140

(22) PCT Filed: Aug. 4, 2020
(86) PCT No.: PCT/CN2020/106696

§ 371 Date: Feb. 14, 2022

(30) Foreign Application Priority Data
Aug. 14, 2019 (CN) 201910748231.3

(51) Int. Cl. C07H 19/10; C12N 15/88; C07H 19/06; C07H 21/02

(52) U.S. Cl. CPC C07H 19/10; C12N 15/88; C07H 19/06; C07H 21/02

(57) ABSTRACT

A modified cytidine compound, that is, an aminooxy group is modified at the 4-position of a cytidine pyrimidine ring to produce derivative cytidine and nucleic acid containing the derivative cytidine, such as RNA. The expression level of the nucleic acid containing the modified cytidine, in particular mRNA, in the body is significantly improved.

### CROSS-REFERENCE TO RELATED APPLICATIONS

This application claims the priority of Chinese prior application with application No.201910748231.3, filed on Aug. 14, 2019.

### TECHNICAL FIELD

The present application relates to the biological field, and in particular to a modified nucleoside and a method for synthesizing the same.

### BACKGROUND

Messenger RNA (mRNA) plays a vital role in human biology. Through a process known as transcription, mRNA controls the protein synthesis in human body. mRNA drugs can be used for genetic diseases, cancers and infectious diseases.

Naturally occurring RNA is synthesized from four basic ribonucleotides ATP, CTP, UTP and GTP, but may contain post-transcriptionally modified nucleotides. Nearly one hundred different modified nucleosides have been identified in RNA (Rozenski, J, Crain, P, and McCloskey, J. (1999). The RNA Modification Database: 1999 update.Nucl Acids Res 27: 196-197). However, many of the modifications, when incorporated into a mRNA, cause an inhibitory immune response and/or limit the protein production in the recipient, thus limiting the therapeutic benefit of the mRNA drug. Therefore, there is a need in the field for novel modified nucleosides, nucleotides, and/or nucleic acids (e.g., mRNA) to address these problems.

### SUMMARY

Provided herein are compounds, modified nucleosides, modified nucleotides, modified nucleic acids, and methods for synthesizing the same.

In one aspect, provided is a compound of Formula (I):

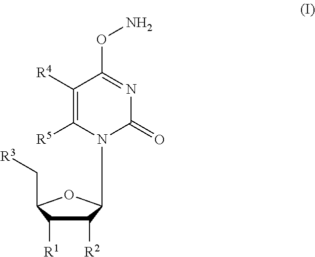

or a pharmaceutically acceptable salt thereof, wherein: R¹, R², R⁴, and R⁵ are each independently selected from the group consisting of —H, —OH, —NH₂, halo, substituted or unsubstituted C₁-C₁₀ alkyl, substituted or unsubstituted aryl, substituted or unsubstituted heteroaryl, substituted or unsubstituted C₁-C₁₀ aralkyl, substituted or unsubstituted C₁-C₁₀ cycloalkyl, substituted or unsubstituted C₁-C₁₀ heterocyclyl, substituted or unsubstituted acyl, —OR⁶, —C(O)R⁶, —C(O)—O—R⁶, —C(O)—NH—R⁶, and —N(R⁶)₂; R³ is selected from the group consisting of —H, —OH, —NH₂, halo, substituted or unsubstituted C₁-C₁₀ alkyl, substituted or unsubstituted aryl, substituted or unsubstituted heteroaryl, substituted or unsubstituted C₁-C₁₀aralkyl, substituted or unsubstituted C₁-C₁₀ cycloalkyl, substituted or unsubstituted C₁-C₁₀ heterocyclyl, substituted or unsubstituted acyl, —OR⁶, —C(O)R⁶, —C(O)—O—R⁶, —C(O)—NH—R⁶, and —N(R⁶)₂, phosphate, diphosphate, and triphosphate; and R⁶ is —H, substituted or unsubstituted C₁-C₁₀ alkyl, and substituted or unsubstituted acyl.

In some examples, R¹, R², R⁴, and R⁵ are each independently —H, —OH, or substituted or unsubstituted C₁-C₁₀ alkyl. In some examples, R³ is —H, —OH, substituted or unsubstituted alkyl, phosphate, diphosphate, or triphosphate. In some examples, R¹ is —OH. In some examples, R² is —OH or —OCH₃. In some examples, R² is —OH. In one example, R² is —OCH₃. In some examples, R³ is —OH. In some examples, R⁴ is —H. In some examples, R⁵ is —H.

In some examples, the compound is a modified nucleoside, wherein R¹ is —OH, R² is —OH and R³ is ——OH. For example, the compound may have the structure of Formula (I-a):

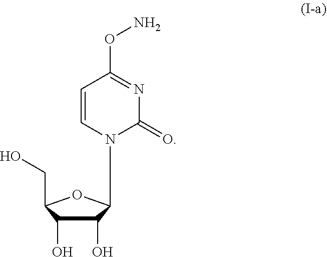

In some examples, the compound is a modified nucleoside, wherein R¹ is —OH, R² is —H and R³ is —OH. For example, the compound may have the structure of Formula (I-b):

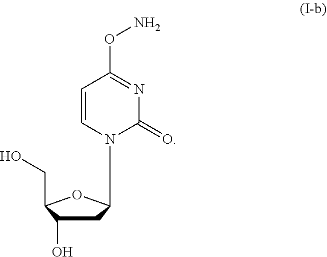

In some examples, the compound is a modified nucleotide, wherein R² is —OH and R³ is phosphate. For example, the compound may have the structure of Formula (I-c):

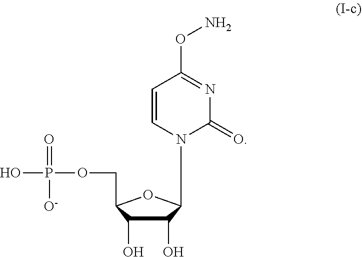

In some examples, the compound is a modified nucleotide, wherein R² is —H and R³ is phosphate. For example, the compound may have the structure of Formula (I-d):

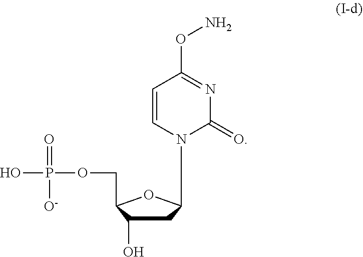

In some examples, the compound is a modified nucleotide, for example, a modified nucleoside triphosphate (NTP), wherein R² is —OH and R³ is triphosphate. For example, the compound may have the structure of Formula (I-e):

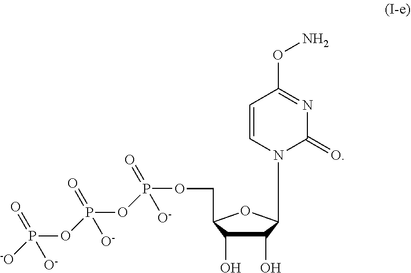

In some examples, the compound is a modified nucleotide, for example, a modified nucleoside triphosphate (NTP),wherein R² is —H and R³ is triphosphate. For example, the compound may have the structure of Formula (I-f):

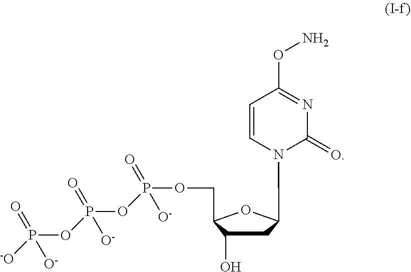

In another aspect, provided is a modified nucleoside triphosphate (NTP) having the structure of Formula (I-g):

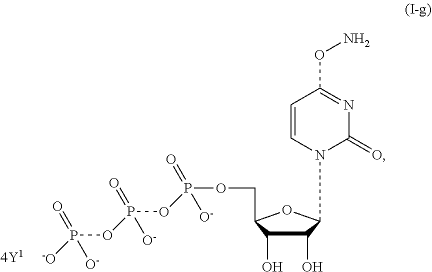

wherein, Y⁺ is a cation.

In some examples, the modified nucleoside triphosphate comprises a modified cytidine triphosphate. In some examples, the Y⁺ is selected from the group consisting of Li⁺, Na⁺, K⁺, H⁺, NH₄⁺ and tetraalkylammonium (NH₄⁺, wherein R is alkyl). In some examples, the tetraalkylammonium is selected from the group consisting of tetraethylammonium, tetrapropylammonium and tetrabutylammonium. In some examples, the tetraallkylammonium is NIR₄⁺, wherein R is alkyl. In some examples, the NR₄⁺ is selected from the group consisting of N(ethyl)₄⁺, N(n-propyl)₄⁺, and N (n-butyl)₄⁺.

In another aspect, provided is a modified deoxynucleoside triphosphate (dNTP) having the structure of Formula (I-h):

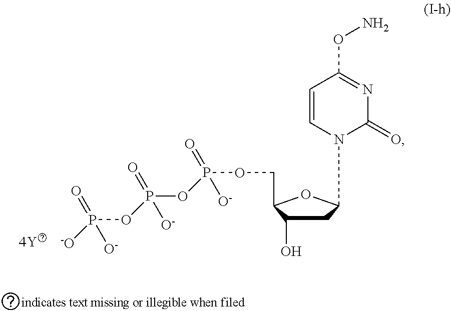

wherein, Y⁺ is a cation.

In some examples, the modified nucleoside triphosphate comprises a modified deoxycytidine triphosphate. In some examples, the Y+is selected from the group consisting of Li⁺, Na⁺, K⁺, H⁺, NH₄⁺ and tetraalkylammonium (NH₄⁺, wherein R is alkyl). In some examples, the tetraalkylammonium is selected from the group consisting of tetraethylammonium, tetrapropylammonium, and tetrabutylammonium. In some examples, the tetraallkylammonium is NIR₄⁺, wherein R is alkyl. In some examples, the NR₄⁺ is selected from the group consisting of N(ethyl)₄⁺, N(n-propyl)₄⁺, and N (n-butyl)₄⁺.

In another aspect, provided is a nucleic acid comprising two or more covalently bonded nucleotides, wherein at least one of the two or more covalently bonded nucleotides comprises any compound, modified nucleoside or modified nucleotide disclosed herein. In some examples, the nucleic acid is a ribonucleic acid (RNA). In some examples, the RNA comprises any compound, modified nucleoside or modified nucleotide disclosed herein. In some examples, the RNA is a messenger RNA (mRNA). In some examples, the nucleic acid is a deoxyribonucleic acid (DNA). In some examples, the DNA comprises any compound, modified nucleoside or modified nucleotide disclosed herein.

In another aspect, provided is a pharmaceutical composition, comprising any compound, modified nucleoside, or modified nucleotide disclosed herein, or a pharmaceutically acceptable salt thereof, and a pharmaceutically acceptable excipient. In some examples, the pharmaceutical composition comprises any compound disclosed herein, or a pharmaceutically acceptable salt thereof; and a pharmaceutically acceptable excipient. In some examples, the pharmaceutical composition comprises any nucleic acid disclosed herein, or a pharmaceutically acceptable salt thereof; and a pharmaceutically acceptable excipient. In some examples, the pharmaceutical composition comprises any RNA disclosed herein, or a pharmaceutically acceptable salt thereof; and a pharmaceutically acceptable excipient. In some examples, the pharmaceutical composition comprises any mRNA disclosed herein, or a pharmaceutically acceptable salt thereof; and a pharmaceutically acceptable excipient.

In another aspect, provided is a compound of Formula (II):

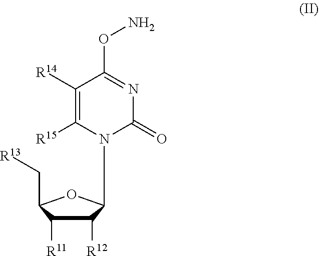

or a pharmaceutically acceptable salt thereof, wherein, R¹¹, R¹², and R¹³ are each independently—H, —OH, —OCH₃ or —O— protecting group; and R¹⁴ and R¹⁵ are each independently selected from —H, substituted or unsubstituted C₁-C₁₀ alkyl and substituted or unsubstituted acyl.

In some examples, R¹¹, R¹² and R¹³ are a —O-protecting group. In some examples, the protecting group is selected from the group consisting of acetyl, benzoyl, benzyl, β-methoxyethoxymethyl, dimethoxytrityl [bis-(4-methoxyphenyl)phenylmethyl], methoxy, methoxytrityl [(4-methoxyphenyl)diphenylmethyl], p-methoxybenzyl, methylthiomethyl, pivaloyl, tetrahydropyranyl, tetrahydrofuranyl, trityl (triphenylmethyl), silyl, methyl, and ethoxyethyl. In some examples, the protecting group is a silyl selected from the group consisting of trimethylsilyl (TMS), tert-butyldiphenylsilyl (TBDPS), tert-butyldimethylsilyl chloride (TBDMS), and triisopropylsilyl (TIPS). In some examples, the protecting group is TB.

In some examples, R¹⁴ and R¹⁵ are —H. In some examples, the compound has the following structure:

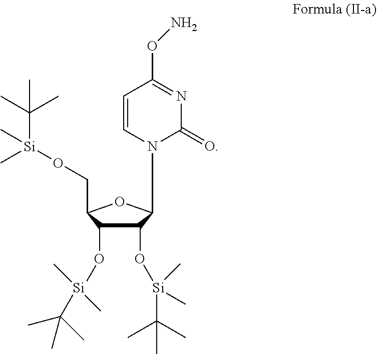

In another aspect, provided is a method for preparing a compound of Formula (I-a) or Formula (I-b), comprising: contacting a compound of Formula (III) with a deprotecting agent,

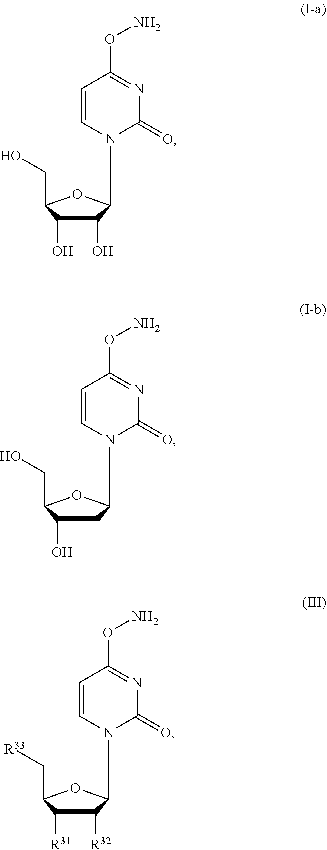

wherein: R³¹ and R³³ are each independently —O— protecting group; and R³² is —H or —O— protecting group. In some examples, the deprotecting agent is selected from the group consisting of tetra-n-butylammonium fluoride (TBAF), tris(dimethylamino)sulfonium difluorotrimethylsilicate (TASF), hydrochloric acid (HCl), camphorsulfonic acid, Pyr·TsOH, Pyr·HF, BF₃·OEt₂, AcOH, LiBF₄, Et₃N·3HF, Et₃NBn+ClKF·2H₂O, and any combination thereof. In some examples, the deprotecting agent is TBAF or Et₃N·3HF. In some examples, the contacting is implemented in presence of an organic solvent. In some examples, the organic solvent is selected from the group consisting of tetrahydrofuran (THF), methanol, ethanol, methylene dichloride, dimethylformamide (DMF), acetonitrile, and any combination thereof. In some examples, the organic solvent is THF.

In some examples, the method further comprises contacting a compound of Formula (III-a) or (III-b) with potassium tert-butoxide, O-(mesitylene sulfonyl) hydroxylamine (MSH) or any combination thereof to form the compound of Formula (III),

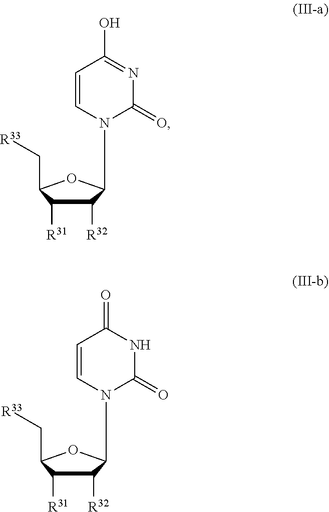

wherein: R³¹ and R³³ are each independently —O— protecting group; and R³² is —H or —O— protecting group. In some examples, the contacting is implemented in presence of methanol, dichloromethane or any combination thereof.

In some examples, the method further comprises contacting uridine or deoxyuridine with tert-butyldimethylsilyl chloride to form the compound of Formula (III-a) or Formula (III-b). In some examples, the contacting is implemented in presence of imidazole, CH₂Cl₂, pyridine, DMF, trimethylamine, DMSO, NaHCO₃, or any combination thereof. For example, the contacting is implemented in presence of DMF. In some examples, the protecting group is selected from the group consisting of acetyl, benzoyl, benzyl, β-methoxyethoxymethyl, dimethoxytrityl [bis-(4-methoxyphenyl)phenylmethyl], methoxymethyl, methoxytrityl [(4-methoxyphenyl)diphenylmethyl], p-methoxybenzyl, methylthiomethyl, pivaloyl, tetrahydropyranyl, tetrahydrofuranyl, trityl (triphenylmethyl), silyl, methyl, and ethoxyethyl. In some examples, the protecting group is a silyl selected from the group consisting of trimethylsilyl(TMS), ted-butyldiphenylsilyl(TBDPS), tert-butyldimethylsilylchloride(TBDMS), triisopropylsilyl(TIPS), and any combination thereof. In some examples, the protecting group is TBDMS.

In another aspect, provided is a method for preparing a compound of Formula (I-a), comprising: (a) contacting uridine with tert-butyldimethylsilyl chloride to form a compound of Formula (II-b) or Formula (II-c):

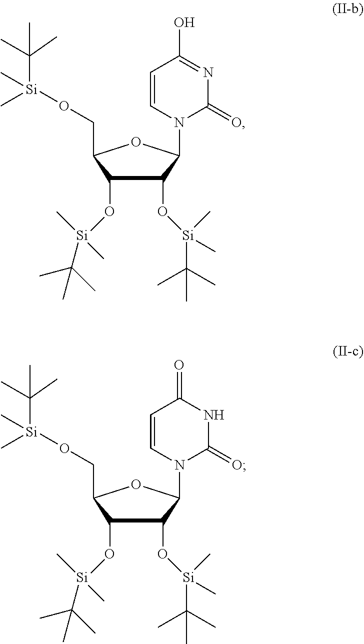

(b) contacting the compound of Formula (II-b) or Formula (II-c) with potassium tert-butoxide and O-(mesitylene sulfonyl)hydroxylamine (MSH) to form a compound of Formula (II-a):

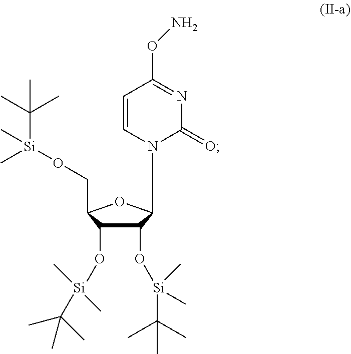

(c) contacting the compound of Formula (II-a) with tetra-n-butylammonium fluoride (TBAF) to form a compound of Formula (I-a): (I-a).

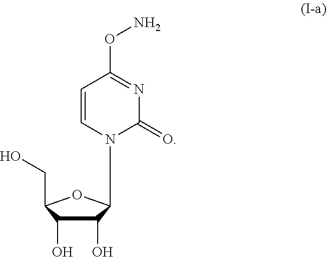

All publications, patents, and patent applications mentioned in this description are herein incorporated by reference to the same extent as if each individual publication, patent, or patent application was specifically and individually indicated to be incorporated by reference. Provided herein are compounds, modified nucleosides, modified nucleotides, modified nucleic acids, and synthesis thereof.

The provided may be a compound of Formula (I):

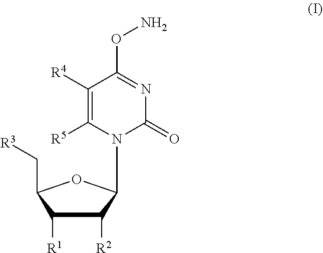

or a pharmaceutically acceptable salt thereof, wherein: R¹, R², R⁴, and R⁵ are each independently selected from the group consisting of —H, —OH, —NH₂, halo, substituted or unsubstituted C₁-C₁₀ alkyl, substituted or unsubstituted aryl, substituted or unsubstituted heteroaryl, substituted or unsubstituted C₁-C₁₀ aralkyl, substituted or unsubstituted C₁-C₁₀ cycloalkyl, substituted or unsubstituted C₁-C₁₀ heterocyclyl, substituted or unsubstituted acyl, —OR⁶, —C(O)R⁶, —C(O)—O—R⁶, —C(O)—NH—R⁶, and —N(R⁶)₂; R³ is selected from the group consisting of —H, —OH, —NH₂, halo, substituted or unsubstituted C₁-C₁₀ alkyl, substituted or unsubstituted aryl, substituted or unsubstituted heteroaryl, substituted or unsubstituted C₁-C₁₀ aralkyl, substituted or unsubstituted C₁-C₁₀ cycloalkyl, substituted or unsubstituted C₁-C₁₀ heterocyclyl, substituted or unsubstituted acyl, —OR⁶, —C(O)R⁶, —C(O)—O—R⁶, —C(O)—NH—R⁶, and —N(R⁶)₂, phosphate, diphosphate and triphosphate; and R⁶ is each independently —H, substituted or unsubstituted C₁-C₁₀ alkyl, and substituted or unsubstituted acyl. The compounds of Formula (I) may exist in different tautomeric forms, and all such forms are embraced within the scope of the present disclosure.

In some examples, R¹, R², and R³ are —OH; R⁴ and R⁵ are —H. The compound can be a modified nucleoside, for example, a modified uridine or modified cytidine (e.g., 4-aminooxycytidine). As shown in FIG. 1, the 4-aminooxycytidine may be produced using the following synthesis scheme:

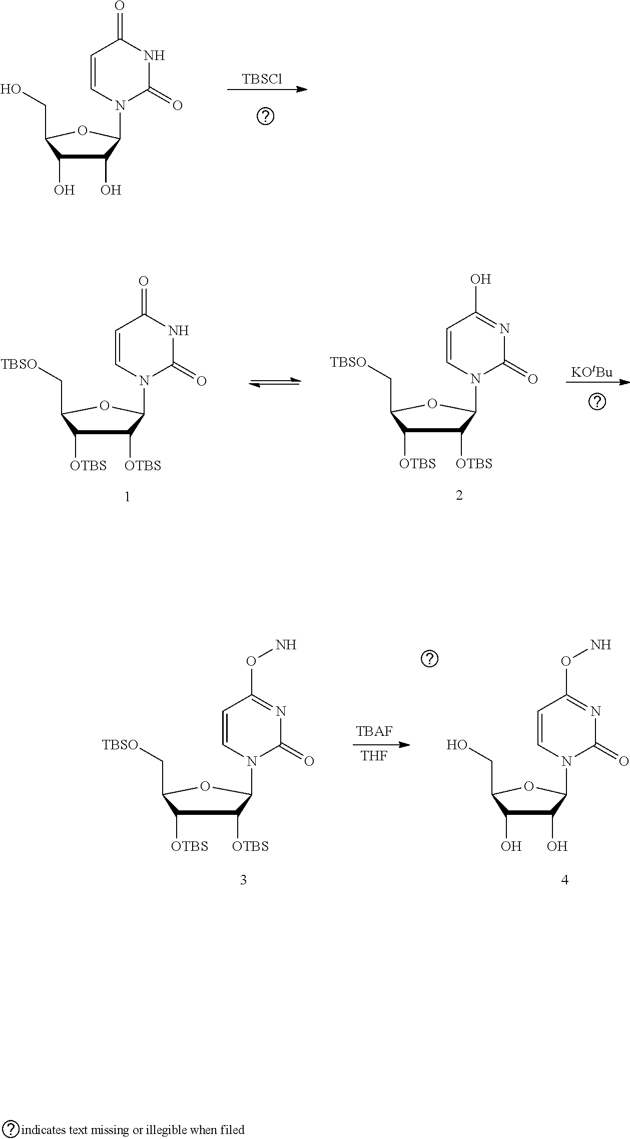

Further provided is a compound of Formula (IV):

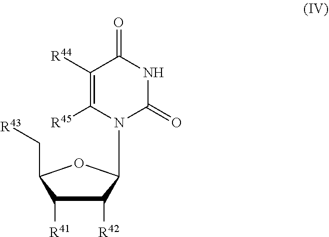

or a pharmaceutically acceptable salt thereof, wherein:

R⁴¹, R⁴² and R⁴³ are each independently —H or —O— protecting group; R⁴⁴ and R⁴⁵ are each independently selected from —H, substituted or unsubstituted C₁-C₁₀alkyl, and substituted or unsubstituted acyl.

Further provided is a compound of Formula (IV-a):

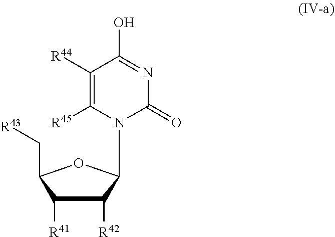

or a pharmaceutically acceptable salt thereof, wherein, R⁴¹, R⁴² and R⁴³ are each independently —H or —O— protecting group; R⁴⁴ and R⁴⁵ are each independently selected from —H, substituted or unsubstituted C₁-C₁₀ alkyl, and substituted or unsubstituted acyl.

In some examples, the compound of Formula (IV-a) and the compound of Formula (IV) are tautomers:

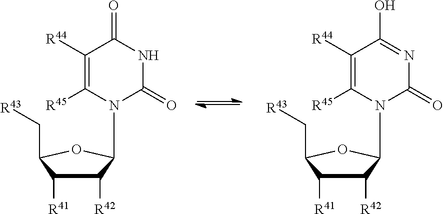

In some examples, the compound of Formula (IV) or Formula (IV-a) may be produced by contacting substituted or unsubstituted uridine or deoxyuridine with a protecting agent via the following synthetic scheme, wherein, R⁴¹ and R⁴³ are each independently —O— protecting group, and R⁴² is —H or —O— protecting group:

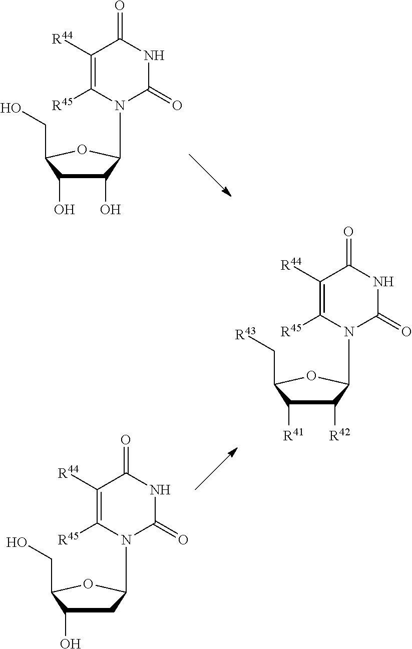

In some examples, the protecting group is selected from the group consisting of acetyl, benzoyl, benzyl, β-methoxyethoxymethyl, dimethoxytrityl [bis-(4-methoxyphenyl)phenylmethyl], methoxymethyl, methoxytrityl [(4-methoxyphenyl)diphenylmethyl], p-methoxybenzyl, methylthiomethyl, pivaloyl, tetrahydropyranyl, tetrahydrofuranyl, trityl (triphenylmethyl), silyl, methyl, and ethoxyethyl. In some examples, the protecting group is a silyl selected from the group consisting of trimethylsilyl (TMS), tert-butyldiphenylsilyl (TBDPS), tert-butyldimethylsilyl chloride (TBDMS), and triisopropylsilyl (TIPS). In some examples, the protecting group is TBDMS. The protecting agents used for producing the protecting groups can be found in the Organic Synthesis Archive (https://www.synarchive.com/protecting-group). In some examples, the protecting agent is tert-butyldimethylsilyl chloride.

Further provided is a compound of Formula (IV-b):

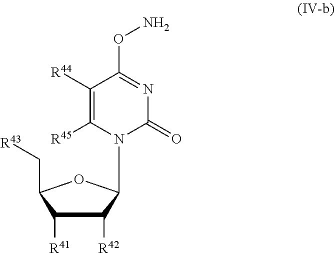

or a pharmaceutically acceptable salt thereof, wherein:

R⁴¹ and R⁴³ are each independently —O— protecting group;

and R⁴² is —H or —O— protecting group; and

R⁴⁴ and R⁴⁵ are each independently —H, substituted or unsubstituted C₁-C₁₀ alkyl, and substituted or unsubstituted acyl.

In some examples, the compound of Formula (IV-b) may be produced by contacting the compound of Formula (IV) or Formula (IV-a) with potassium tert-butoxide and/or O-(mesitylene sulfonyl)hydroxylamine (MSH) via the following synthetic scheme:

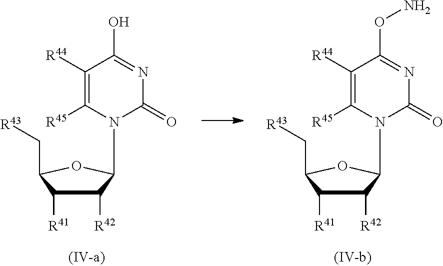

Further provided is a compound of Formula (IV-c) or Formula (IV-d):

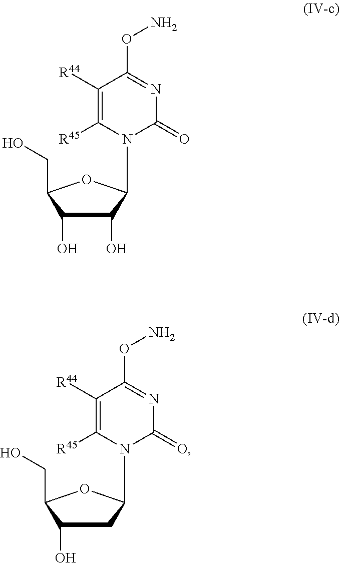

or a pharmaceutically acceptable salt thereof, wherein:

R⁴⁴ and R⁴⁵ are each independently —H, substituted or unsubstituted C₁-C₁₀ alkyl, and substituted or unsubstituted acyl.

In some examples, the compound of Formula (IV-c) or Formula (IV-d) may be produced by contacting the compound of Formula (IV-b) with a deprotecting agent via the following synthetic scheme:

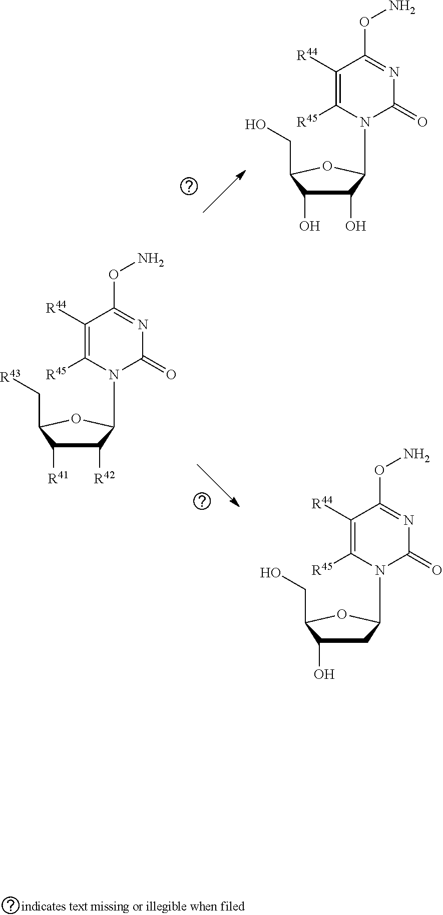

In some examples, the deprotecting agent is selected from the group consisting of tetra-n-butylammonium fluoride (TBAF), tris(dimethylamino)sulfonium difluorotrimethylsilicate (TASF), hydrochloric acid (HCl), camphorsulfonic acid, Pyr·TsOH, Pyr·HF, BF₃·OEt₂, AcOH, LiBF₄, Et₃N·3HF, Et₃NBn⁺ClKF·2H₂O, and any combination thereof. In some examples, the deprotecting group is TBAF. In some examples, the deprotecting group is Et₃N·3HF.

### BRIEF DESCRIPTION OF THE DRAWINGS

FIG. 1 shows a synthesis scheme used for synthesizing the modified nucleotide (e.g., 4-aminooxycytidine and 4-hydroxylaminecytosine).

FIG. 2 shows a comparative experiment of the expression between several modified cytidines according to the present disclosure and several cytidines of the existing control.

FIG. 3 shows a comparative experiment of cell expression of a Poly structure in several modified structures according to the present disclosure.

FIG. 4 is a graph decipting the experimental result of cell expression of cytidines with various forms of modification according to the present disclosure at various modification rates (a specific structure of Invention 1).

FIG. 5 is a graph decipiting the experimental result of cell expression of cytidines with various forms of modification according to the present disclosure at various modification rates (a specific structure of Invention 4).

FIG. 6 is a graph decipiting the experimental result of cell expression of cytidines with various forms of modification according to the present disclosure at various modification rates (a specific structure of Invention 3).

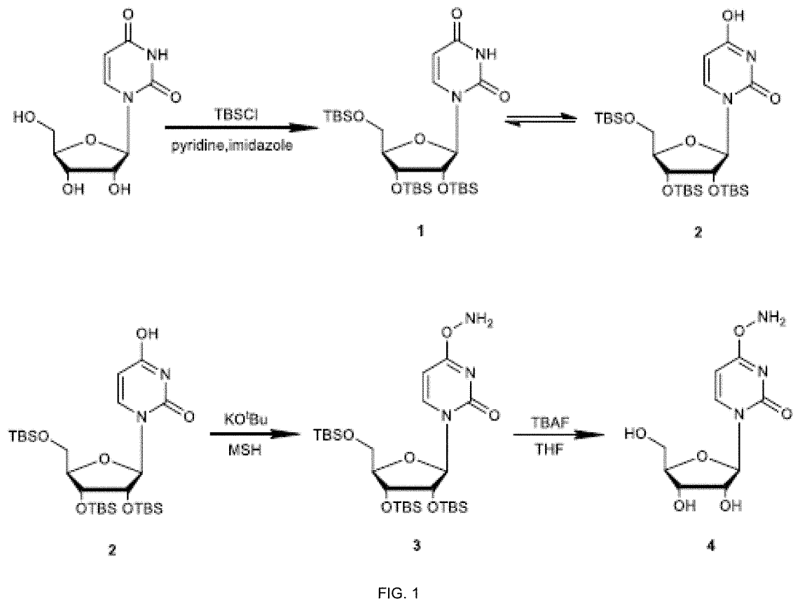

### DETAILED DESCRIPTION OF THE EMBODIMENTS

Definitions

Unless defined otherwise, all technical and scientific terms used herein have the same meaning as commonly understood by one of the ordinary skill in the art to which this disclosure belongs. Although any methods and materials similar or equivalent to those described herein can be used in the practice or testing of the formulations or unit doses herein, some methods and materials are now described. Unless mentioned otherwise, the techniques employed or contemplated herein are standard methodologies. The materials, methods and examples are illustrative only and not limiting.

As used herein and in the appended claims, the singular forms “a/an,” “one,” and “the” include plural referents unless the context clearly dictates otherwise. Thus, for example, reference to “a compound” includes at least two such agents, and reference to “the salt” includes reference to one or more salts (or to at least two salts) and equivalents thereof known to those skilled in the art, and so forth.

Unless otherwise indicated, some embodiments herein contemplate numerical ranges. When a numerical range is provided, unless otherwise indicated, the range includes the range endpoints. Unless otherwise indicated, numerical ranges include all values and sub-ranges therein as if explicitly written out. For example, the term “C₁-C₁₀ alkyl” (or interchangeable referred as C₁-C₁₀ alkyl) is specifically intended to individually disclose methyl, ethyl, C₃ alkyl, C₄ alkyl, C₅ alkyl, C₆ alkyl, C₇ alkyl, C₈ alkyl, C₉ alkyl, and C₁₀ alkyl.

The term “optional” or “optionally” means that a subsequently described event or circumstance may or may not occur and that the description includes instances when the event or circumstance occurs and instances in which it does not. For example, “optionally substituted aryl” means that the aryl radical may or may not be substituted and that the description includes both substituted aryl radicals and unsubstituted aryl radicals.

The term “substituted” may refer to a radical in which one or more hydrogen atoms are each independently replaced with the same or different substituent(s). Typical substituents include, but are not limited to, halo, alkyl, aryl, aralkyl, cycloalkyl, or acyl.

The term “about” and its grammatical equivalents in relation to a reference numerical value and its grammatical equivalents as used herein can include a range of values plus or minus 10% from that value, such as a range of values plus or minus 10%, 9%, 8%, 7%, 6%, 5%, 4%, 3%, 2%, or 1% from that value. For example, the amount “about 10” includes amounts from 9 to 11.

The term “comprising” (and related terms such as “comprise” or “comprises” or “having” or “including”) is not intended to exclude that in other certain embodiments, for example, an embodiment of any composition, method, or process, or the like, described herein, may “consist of” or “consist essentially of” the described features.

The term “compound” of the present application includes solvates, esters and prodrugs thereof. The compounds disclosed herein may exist in different tautomeric forms, and all such forms are encompassed within the scope of the present disclosure. The compounds disclosed herein may contain one or more asymmetric centers and may thus give rise to enantiomers, diastereomers, and other stereoisomeric forms that may be defined, in terms of absolute stereochemistry, as (R) or (S). Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.). Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring. The compounds of the present application optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. All isotopic variations of the compounds according to the present disclosure, whether radioactive or not, are encompassed within the scope of the present disclosure. In certain embodiments, the compounds disclosed herein have some or all of the ¹H atoms replaced with ²H atoms. The methods of synthesis for deuterium-containing substituted heterocyclic derivative compounds are known in the art and include, by way of non-limiting example only, the following synthetic methods. Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The term “solvate” can include, but is not limited to, a solvate that retains one or more of the activities and/or properties of the compound and that is not undesirable. Examples of solvates include, but are not limited to, a compound in combination with water, isopropanol, ethanol, methanol, DMSO, ethyl acetate, acetic acid, ethanolamine, or combinations thereof.

The term “solvent” may include, but is not limited to, non-polar, polar aprotic, polar protic solvents, and ionic liquids. Illustrative examples of non-polar solvents include, but are notlimited to, pentane, cyclopentane, hexane, cyclohexane, benzene, toluene, 1,4-dioxane, chloroform, diethyl ether, and dichloromethane (DCM). Illustrative examples of polar aprotic solvents include, but are not limited to, tetrahydrofuran (THF), ethyl acetate, acetone, dimethylformamide (DMF), acetonitrile (MeCN), dimethyl sulfoxide (DMSO), nitromethane, and propylene carbonate. Illustrative examples of polar protic solvents include, but are not limited to, formic acid, n-butanol, isopropanol (IPA), n-propanol, ethanol, methanol, acetic acid, and water. Illustrative examples of ionic liquids include, but are not limited to, 1-alkyl-3-methylimidazolium cations, 1-alkylpyridinium cations, N-methyl-N-alkylpyrrolidinium cations, 1-butyl-3-methylimidazolium tetrachloroferrate, 1-butyl-3-methylimidazolium chloride, and tetraalkylphosphonium iodide.

The term “tautomer” refers to a molecule wherein a proton may shift from one atom of a molecule to another atom of the same molecule. The compounds presented herein may, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

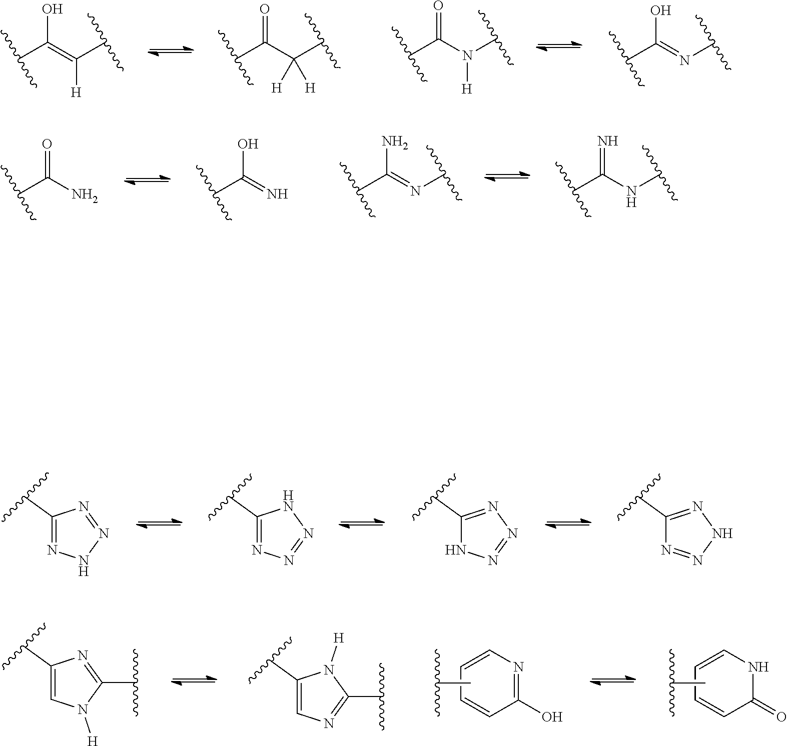

The term “ester” refers to a derivative from an acid in which at least one —OH (hydroxyl) group is replaced by an —O— alkyl (alkoxy) group.

The term “prodrug” is meant to indicate a compound that may be converted under physiological conditions or by solvolysis to a biologically active compound described herein. Thus, the term “prodrug” refers to a precursor of a biologically active compound that is pharmaceutically acceptable. A prodrug may be inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam).

The term “protecting group” refers to a group of atoms that mask, reduce or prevent the reactivity of the functional group when attached to a reactive functional group in a molecule. Typically, a protecting group may be selectively removed as desired during the course of a synthesis. Examples of protecting groups can be found in Wuts and Greene, “Greene's Protective Groups in Organic Synthesis,” 4th Ed., Wiley Interscience (2006), and Harrison et al., Compendium of Synthetic Organic Methods, Vols. 1-8, 1971-1996, John Wiley & Sons, NY. Functional groups that may have a protecting group include, but are not limited to, hydroxy, amino, and carboxy groups. Representative amine protecting groups include, but are not limited to, formyl, acetyl (Ac), trifiuoroacetyl, benzyl (Bn), benzoyl (Bz), carbamate, benzyloxycarbonyl (“CBZ”), p-methoxybenzyl carbonyl (Moz or MeOZ), tertbutoxycarbonyl (“Boc”), trimethylsilyl (“TMS”), 2-trimethylsilyl-ethanesulfonyl (“SES”), trityl and substituted trityl groups, allyloxycarbonyl, 9-fluorenylmethyloxycarbonyl (“FMOC”), nitro-veratryloxycarbonyl (“NVOC”), p-methoxybenzyl (PMB), tosyl (Ts) and the like.

The term “salt” is intended to include, but not be limited to, pharmaceutically acceptable salts. And the term “pharmaceutically acceptable salt” is intended to mean those salts that retain one or more of the biological activities and properties of the free acids and bases and that are not biologically or otherwise undesirable. Illustrative examples of pharmaceutically acceptable salts include, but are not limited to, sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanone, propiolates, oxalates, malonates, succinates, suberates, sebacates, fumarates, maleates, butyne-1,4-dioates, hexyne-1,6-dioates, benzoates, chlorobenzoates, methyl benzoates, dinitrobenzoates, hydroxybenzoates, methoxybenzoates, phthalates, sulfonates, xylene, phenylacetic acid, phenylbutyrates, citrates, lactates, γ-hydroxybutyrates, glycolates, tartrates, methanesulfonates, propanesulfonates, naphthalene-1-sulfonates, naphthalene-2-sulfonates, and mandelates.

The term “acid” refers to molecules or ions capable of donating a hydron (proton or hydrogen ion H⁺), or, alternatively, capable of forming a covalent bond with an electron pair (e.g., a Lewis acid). Acids can include, but not limited to, inorganic acids, sulfonic acids, carboxylic acids, halogenated carboxylic acids, vinylogous carboxylic acids, and nucleic acids. Illustrative examples of inorganic acids, but are not limited to, hydrogen halides and their solutions: hydrofluoric acid (HF), hydrochloric acid (HCl), hydrobromic acid (HBr), hydroiodicacid (HI); halogen oxoacids: hypochlorous acid (HClO), chlorous acid (HClO₂), chloric acid (HClO₃), perchloric acid (HClO₄), and corresponding analogs for bromine and iodine, and hypofluorous acid (HFO); sulfuric acid (H₂SO₄); fluorosulfuric acid (HSO₃F); nitricacid (HNO₃); phosphoric acid (H₃PO₄); fluoroantimonic acid (HSbF₆); fluoroboric acid (HBF₄); hexafluorophosphoric acid (HPF₆); chromic acid (H₂CrO₄); and boric acid (H₃BO₃). Illustrative examples of sulfonic acids include, but are not limited to, methanesulfonic acid (or mesylic acid, CH₃SO₃H), ethanesulfonic acid (or esylic acid, CH₃CH₂SO₃H), benzenesulfonic acid (or besylic acid, C₆H₅SO₃H), p-toluenesulfonic acid (or tosylic acid, CH₃C₆H₄SO₃H), trifluoromethanesulfonic acid (or triflic acid, CF₃SO₃H), and polystyrene sulfonicacid (sulfonated polystyrene, [CH₂CH(C₆H₄)SO₃H]ₙ). Illustrative examples of carboxylic acids include, but are not limited to, acetic acid (CH₃COOH), citric acid (C₆H₈O₇), formicacid (HCOOH), gluconic acid (HOCH₂—(CHOH)₄—COOH), lactic acid (CH₃—CHOH—COOH), oxalic acid (HOOC—COOH), and tartaric acid (HOOC—CHOH—CHOH—COOH). Illustrative examples of halogenated carboxylic acids include, but are not limited to, fluoroacetic acid, trifluoroacetic acid, chloroacetic acid, dichloroacetic acid, and trichloroacetic acid. Illustrative examples of vinylogous carboxylic acids include, but are not limited to, ascorbic acid. Illustrative examples of nucleic acids include, but are not limited to, deoxyribonucleic acid (DNA) and ribonucleic acid (RNA).

The term “base” refers to molecules or ions capable of accepting protons from a proton donor and/or produce hydroxide ions (OH⁻). Illustrative examples of bases include, but are not limited to, aluminum hydroxide (Al(OH)₃), ammonium hydroxide (NH₄OH), arsenic hydroxide (As(OH)₃), barium hydroxide (Ba(OH)₂), beryllium hydroxide (Be(OH)₂), bismuth(III) hydroxide (Bi(OH)₃), boron hydroxide (B(OH)₃), cadmium hydroxide (Cd(OH)₂), calcium hydroxide (Ca(OH)₂), cerium(III) hydroxide (Ce(OH)₃), cesium hydroxide (CsOH), chromium(II) hydroxide (Cr(OH)₂),chromium(III) hydroxide (Cr(OH)₃), chromium(V) hydroxide (Cr(OH)₅), chromium(VI) hydroxide (Cr(OH)₆), cobalt(II) hydroxide (Co(OH)₂), cobalt(III) hydroxide (Co(OH)₃), copper(I) hydroxide (CuOH), copper(II) hydroxide (Cu(OH)₂), gallium(II) hydroxide (Ga(OH)₂), gallium(III) hydroxide (Ga(OH)₃), gold(I) hydroxide (AuOH), gold(III) hydroxide (Au(OH)₃), indium(I) hydroxide (InOH), indium(II) hydroxide (In(OH)₂), indium(III) hydroxide (In(OH)₃), iridium(III) hydroxide (Ir(OH)₃), iron(II) hydroxide (Fe(OH)₂), iron(III) hydroxide (Fe(OH)₃), lanthanum hydroxide (La(OH), lead(II) hydroxide (Pb(OH)₂), lead(IV) hydroxide (Pb(OH)₄), lithium hydroxide (LiOH), magnesium hydroxide (Mg(OH)₂), manganese(II) hydroxide (Mn(OH)₂), manganese(III) hydroxide (Mn(OH)₃), manganese(IV) hydroxide (Mn(OH)₄), manganese(VII) hydroxide (Mn(OH)₇), mercury(I) hydroxide (Hg₂(OH)₂), mercury(II) hydroxide (Hg(OH)₂), molybdenum hydroxide (Mo(OH)₃),neodymium hydroxide (Nd(OH)₃), nickel oxo-hydroxide (NiOOH), nickel(II) hydroxide (Ni(OH)₂), nickel(III) hydroxide (Ni(OH)₃), niobium hydroxide (Nb(OH)₃), osmium(IV) hydroxide (Os(OH)₄), palladium(II) hydroxide (Pd(OH)₂), palladium(IV) hydroxide (Pd(OH)₄), platinum(II) hydroxide (Pt(OH)₂), platinum(IV) hydroxide (Pt(OH)₄), plutonium(IV) hydroxide (Pu(OH)₄), potassium hydroxide (KOH), radium hydroxide (Ra(OH)₂), rubidium hydroxide (RbOH), ruthenium(III) hydroxide (Ru(OH)₃), scandium hydroxide (Sc(OH)₃), siliconhydroxide (Si(OH)₄), silver hydroxide (AgOH), sodium hydroxide (NaOH), strontium hydroxide (Sr(OH)₂), tantalum(V) hydroxide (Ta(OH)₅), technetium(II) hydroxide (Tc(OH)₂), tetramethylammonium hydroxide (C₄H₁₂NOH), thallium(I) hydroxide (TIOH), thallium(III) hydroxide (TI(OH)₃), thorium hydroxide (Th(OH)₄), tin(II) hydroxide (Sn(OH)₂), tin(IV) hydroxide (Sn(OH)₄), titanium(II) hydroxide (Ti(OH)₂), titanium(III) hydroxide (Ti(OH)₃), titanium(IV) hydroxide (Ti(OH)₄), tungsten(II) hydroxide (W(OH)₂), uranyl hydroxide ((UO₂)₂(OH)₄), vanadium(II) hydroxide (V(OH)₂), vanadium(III) hydroxide (V(OH)₃), vanadium(V) hydroxide (V(OH)₅), ytterbium hydroxide (Yb(OH)₃), yttrium hydroxide (Y(OH)₃), zinc hydroxide (Zn(OH)₂), and zirconium hydroxide (Zr(OH)₄).

The term “alkyl” refers to a straight or branched hydrocarbon chain radical consisting solely of carbon and hydrogen atoms, containing no unsaturation, having from one to fifteen carbon atoms (e.g., C₁₋₁₅ alkyl). In certain embodiments, an alkyl comprises one to thirteen carbon atoms (e.g., C₁₋₁₃ alkyl). In certain embodiments, an alkyl comprises one to ten carbon atoms (e.g., C₁₋₁₀ alkyl). In certain embodiments, an alkyl comprises one to eight carbon atoms (e.g., C₁₋₈ alkyl). In other embodiments, an alkyl comprises one to five carbon atoms (e.g., C₁₋₅ alkyl). In other embodiments, an alkyl comprises one to four carbon atoms (e.g., C₁₋₄ alkyl). In other embodiments, an alkyl comprises one to three carbon atoms (e.g., C₁₋₃ alkyl). In other embodiments, an alkyl comprises one to two carbon atoms (e.g., C₁₋₂ alkyl). In other embodiments, an alkyl comprises one carbon atom (e.g., C₁ alkyl). In other embodiments, an alkyl comprises five to fifteen carbon atoms (e.g., C₅₋₁₅ alkyl). In other embodiments, an alkyl comprises five to ten carbon atoms (e.g., C₅₋₁₀ alkyl). In other embodiments, an alkyl comprises five to eight carbon atoms (e.g., C₅₋₈ alkyl). In other embodiments, an alkyl comprises two to five carbon atoms (e.g., C₂₋₅ alkyl). In other embodiments, an alkyl comprises three to five carbon atoms (e.g., C₃₋₅ alkyl). In other embodiments, the alkyl group is selected from methyl, ethyl, 1-propyl (n-propyl), 1-methylethyl(iso-propyl), 1-butyl(n-butyl), 1-methylpropyl (sec-butyl), 2-methylpropyl (iso-butyl), 1,1-dimethylethyl (tent-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximido, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (wherein t is 1 or 2), —S(O)ₜOR^{a} (wherein t is 1 or 2), —S(O)ₜR^{a} (wherein t is 1 or 2) and —S(O)ₜN(R^{a})₂ (wherein t is 1 or 2); wherein each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroaralkyl (optionally substituted with halogen, hydroxy, methoxy, or trifluoromethyl).

The term “aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, wherein at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substitutedheteroaralkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R_{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (wherein t is 1 or 2), —R^{b}—S(O)ₜR^{a} (wherein t is 1 or 2), —R^{b}—S(O)ₜOR^{a} (wherein t is 1 or 2) and —R^{b}—S(O)ₜN(R^{a})₂ (wherein t is 1 or 2), wherein each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroaralkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and wherein each of the above substituents is unsubstituted unless otherwise indicated.

The term “alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)oR^{a}, —C(O)N(R^{a})₂, —N(R^{a})C(O)OR^{a},—OC(O)—N(R^{a})₂, —N(R^{a})C(O)R^{a}, —N(R^{a})S(O)ₜR^{a} (wherein t is 1 or 2), —S(O)ₜOR^{a} (wherein t is 1 or 2), —S(O)ₜR^{a} (wherein t is 1 or 2) and —S(O)ₜN(R^{a})₂ (wherein t is 1 or 2); wherein each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroaralkyl (optionally substituted with halogen, hydroxy, methoxy, or trifluoromethyl). The term “alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl has two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (wherein t is 1 or 2), —S(O)ₜOR^{a} (wherein t is 1 or 2), —S(O)ₜR^{a} (wherein t is 1 or 2) and —S(O)ₜN(R^{a})₂ (wherein t is 1 or 2); wherein each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroaralkyl (optionally substituted with halogen, hydroxy, methoxy, or trifluoromethyl).

The term “alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, butylene, and the like. The alkylene chain is attached to the rest of the molecule through a single bond and to the radical group through a single bond. The points of attachment of the alkylene chain to the rest of the molecule and to the radical group can be through one carbon in the alkylene chain or through any two carbons within the chain. In certain embodiments, an alkylene comprises one to eight carbon atoms (e.g., C₁₋₈ alkylene). In other embodiments, an alkylene comprises one to five carbon atoms (e.g., C₁₋₅ alkylene). In other embodiments, an alkylene comprises one to four carbon atoms (e.g., C₁₋₄ alkylene). In other embodiments, an alkylene comprises one to three carbon atoms (e.g., C₁₋₃ alkylene). In other embodiments, an alkylene comprises one to two carbon atoms (e.g., C₁₋₂ alkylene). In other embodiments, an alkylene comprises one carbon atom (e.g., C₁ alkylene). In other embodiments, an alkylene comprises five to eight carbon atoms (e.g., C₅₋₈ alkylene). In other embodiments, an alkylene comprises two to five carbon atoms (e.g., C₂₋₅ alkylene). In other embodiments, an alkylene comprises three to five carbon atoms (e.g., C₃₋₅ alkylene). Unless stated otherwise specifically in the specification, an alkylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (wherein t is 1 or 2), —S(O)ₜOR^{a} (wherein t is 1 or 2), —S(O)ₜR^{a} (wherein t is 1 or 2) and —S(O)ₜN(R^{a})₂ (wherein t is 1 or 2) wherein each Ra is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroaralkyl (optionally substituted with halogen, hydroxy, methoxy, or trifluoromethyl).

The term “aralkyl” refers to a radical of the formula —R^{c}-aryl, wherein R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

The term “aralkenyl” refers to a radical of the formula —R^{d}-aryl, wherein R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

The term “aralkynyl” refers to a radical of the formula —R^{e}-aryl, wherein R^{e} is an alkynylene chain as defined above. The aryl part of the aralkynyl radical is optionally substituted as described above for an aryl group. The alkynylene chain part of the aralkynyl radical is optionally substituted as defined above for an alkynylene chain.

The term “carbocyclyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In certain embodiments, a carbocyclyl comprises five to seven carbon atoms. The carbocyclyl is attached to the rest of the molecule by a single bond. Carbocyclyl may be saturated, (i.e., containing single C—C bonds only) or unsaturated (i.e., containing one or more double bonds or triple bonds). A fully saturated carbocyclyl radical is also referred to as “cycloalkyl”. Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl”. Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroaralkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (wherein t is 1 or 2), —R^{b}—S(O)ₜR^{a} (wherein t is 1 or 2), —R^{b}—S(O)ₜOR^{a} (wherein t is 1 or 2) and —R^{b}—S(O)ₜN(R^{a})₂ (wherein t is 1 or 2); wherein each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroaralkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and wherein each of the above substituents is unsubstituted unless otherwise indicated.

The term “fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, fluoromethyl-2-fluoroethyl, and the like. The alkyl part of the fluoroalkyl radical may be optionally substituted as defined above for an alkyl group.

The term “halo” or “halogen” refers to bromo, chloro, fluoro or iodo substituents.

The term “heterocyclyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which may include fused or bridged ring systems. The heteroatoms in the heterocyclyl radical may be optionally oxidized. One or more nitrogen atoms, if present, are optionally quaternized. The heterocyclyl radical is partially or fully saturated. The heterocyclyl may be attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroaralkyl, —R^{b}—OR^{a}, —R^{b}—OC(O)—R^{a}, —-R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R_{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (wherein t is 1 or 2), —R^{b}—S(O)ₜR^{a} (wherein t is 1 or 2), —R^{b}—S(O)ₜOR^{a} (wherein t is 1 or 2) and —R^{b}—S(O)ₜN(R^{a})₂ (wherein t is 1 or 2), wherein each R^{a} is independently substituted by one or more substituents selected from hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroaralkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and wherein each of the above substituents is unsubstituted unless otherwise indicated. The term “heterocyclylalkyl” refers to a radical of the formula —R^{c} heterocyclyl, wherein R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

The term “heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical may be a monocyclic, bicyclic, tricyclic or tetracyclic ring system, wherein at least one of the rings in the ring systemis fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxolyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, quinolinyl, cyclopentadieno[d]pyrimidinyl, 6,7-dihydro-5H-cyclopentadieno[4,5]thieno[2,3-d]pyrimidinyl, 5,6-dihydrobenzo[h]quinazolinyl, 5,6-dihydrobenzo[h]quinolinyl, 6,7-dihydro-5H-benzo[6,7]cyclohepta[1,2-c]pyridazinyl, dibenzofuranyl, dibenzothiophenyl, furanyl, furoyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, isoindolyl, indolinyl, isoindolinyl, isoquinolyl, indolyl isoxazolyl, 5,8-methano-5,6,7,8-tetrahydroquinazolinyl, naphthyridinyl, 1,6-naphthyridinonyl, oxadiazolyl, oxoazepinyl, oxazepanyl, oxazepanyl, oxoacyl, epoxynonyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, piperidinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]prolyl, and thiophenyl (i.e. thienyl). Unless stated otherwise specifically in the specification, the term “heteroaryl” is meant to include heteroaryl radicals as defined above that are optionally substituted by alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substitutedheteroaralkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (wherein t is 1 or 2), —R^{b}—S(O)ₜR^{a} (wherein t is 1 or 2), —R^{b}—S(O)ₜOR^{a} (wherein t is 1 or 2) and —R^{b}—S(O)ₜN(R^{a})₂ (wherein t is 1 or 2), wherein each R^{a} is independently substituted by one or more substituents selected from hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroaralkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and wherein each of the above substituents is unsubstituted unless otherwise indicated.

The term “nucleoside” is defined as a compound containing a pentaglucose (ribose or deoxyribose) or a derivative thereof, and an organic base, purine or pyrimidine or a derivative thereof. The nucleosides described herein may be modified nucleosides. For example, nucleoside may be cytidine, deoxycytidine, uridine, deoxyuridine, adenosine, deoxyadenosine, guanosine, deoxyguanosine, thymidine, 5-methyluridine or inosine.

The term “nucleotide” is defined as a nucleoside with the addition of at least one phosphate group. The nucleotides may include a phosphate group, a diphosphate group, or a triphosphate group. In another embodiment, “nucleotides” refer to the monomeric units of nucleic acid polymers. The nucleotides described herein may be modified nucleotides. For example, nucleotides may be nucleoside triphosphate

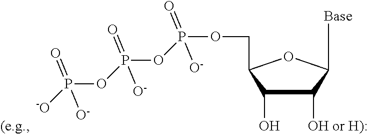

adenosine triphosphate (ATP), guanosine triphosphate (GTP), cytidine triphosphate (CTP), or uridine triphosphate (UTP).

The term “nucleic acid” includes any compound and/or substance that is or may be incorporated into an oligonucleotide chain. Exemplary nucleic acids for use in accordance with the present disclosure include, but are not limited to, DNAs, RNAs including messenger mRNAs (mRNAs), hybrids thereof, RNAi-inducing agents, RNAi agents, siRNAs, shRNAs, miRNAs, antisense RNAs, ribozymes, catalytic DNAs, RNAs that induce triple helix formation, aptamers, vectors, etc., described in detail herein.

The term “deoxyribonucleic acid”, “DNA” or “DNA molecule” refers to a molecule consisting of two chains (polynucleotide), and each chain includes monomeric units of nucleotide. Nucleotides are mutually linked with each other via a covalent bond between sugar of one nucleotide and a phosphate of the following nucleotide, to generate an alternating sugar-phosphate backbone. Nitrogenous bases from the two separated polynucleotide chains are associated through hydrogen bonds to prepare a double-stranded DNA.

The term “ribonucleic acid,” “RNA,” or “RNA molecule” refers to a string of at least 2 base-sugar-phosphate combinations. In an embodiment, the term includes compounds consisting of nucleotides in which the sugar moiety is ribose. In another embodiment, a tail end includes both RNA and RNA derivatives in which the backbone is modified. In an embodiment, RNA may be in the form of a tRNA (transfer RNA), snRNA (small nuclear RNA), rRNA (ribosomal RNA), mRNA (messenger RNA), antisense RNA, small inhibitory RNA (siRNA), micro RNA (miRNA) and ribozymes. The use of siRNA and miRNA has been described (Caudy AA et al., Genes & Devel 16:2491-96 and references cited therein). In addition, these forms of RNA may be single, double, triple, or quadruple stranded. In another embodiment, the term also includes artificial nucleic acids that may contain other types of backbones but the same bases. In another embodiment, the artificial nucleic acid is a PNA (peptide nucleic acid). In another embodiment, PNA contains peptide backbones and nucleotide bases and are able to bind to both DNA and RNA molecules. In another embodiment, the nucleotide is modified oxetane. In another embodiment, the nucleotide is modified by replacement of one or more phosphodiester bonds with a phosphorothioate bond. In another embodiment, the modified nucleic acid contains any other variant of the phosphate backbone of native nucleic acids known in the art. The use of phosphothiorate nucleic acids and PNA are known to those skilled in the art, and are described in, for example, Neilsen P E, Curr Opin Struct Biol 9:353-57; and Raz N K et al Biochem Biophys Res Commun. 297:1075-84. The production and use of nucleic acids is known to those skilled in art and is described, for example, in Molecular Cloning, (2001), Sambrook and Russell, eds. and Methods in Enzymology: Methods for molecular cloning in eukaryotic cells (2003) Purchio and G. C. Fareed. Each nucleic acid derivative represents a separate embodiment of the present disclosure.

The term “derivative” may be used interchangeably with the term “analog”. Compound A can be a derivative or analog of compound B, if 1, 2, 3, 4, 5, 6, 7, 8, 9, or 10 atoms of compound A are replaced by another atom or a functional group (e.g., amino, halo, substituted or unsubstituted alkyl, substituted or unsubstituted aryl, substituted or unsubstituted aralkyl, or substituted or unsubstituted cycloalkyl) to form compound B. The terms “derivative” and “analog” can also be used interchangeably with the term “modified,” for example, if Compound A is a derivative of Compound B, then Compound A is also a modified Compound B.

The term “subject” refers to a mammal that has been or will be the object of treatment, observation or experiment. The term “mammal” is intended to have its standard meaning, and encompasses humans, dogs, cats, sheep, and cows, for example. The methods described herein may be useful in both human therapy and veterinary applications. In some embodiments, the subject is a human.

The term “therapeutically effective amount” of a chemical entity described herein refers to an amount effective, when administered to a human or non-human subject, to provide a therapeutic benefit such as amelioration of symptoms, slowing of disease progression, or prevention of disease.

The term “treating” or “treatment” encompasses administration of at least one compound disclosed herein, or a pharmaceutically acceptable salt thereof, to a mammalian subject, particularly a human subject, in need of such an administration and includes (i) arresting the development of clinical symptoms of the disease, such as cancer, (ii) bringing about a regression in the clinical symptoms of the disease, such as cancer, and/or (iii) prophylactic treatment for preventing the onset of the disease, such as cancer.

Modified Nucleosides

The modified nucleoside may include a compound having the following structure:

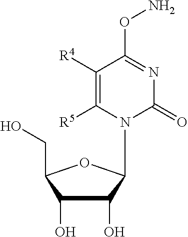

or a pharmaceutically acceptable salt thereof, wherein: R⁴ and R⁵ are each independently selected from H, —OH, —NH₂, halo, substituted or unsubstituted C₁-C₁₀ alkyl, substituted or unsubstituted aryl, substituted or unsubstituted aralkyl, substituted or unsubstituted cycloalkyl, substituted or unsubstituted acyl, —OR⁶, —C(O)R⁶, and —NR⁶; and R⁶ is each independently H, substituted or unsubstituted C₁-C₁₀ alkyl, and substituted or unsubstituted acyl. In some examples, R⁴ is H. In some examples, R⁵ is H. The modified nucleoside may be a modified uridine or cytidine, for example, 4-aminooxycytidine. The modified nucleosides may be the compound of Formula (I-a).

The modified nucleoside may also comprise m¹A (1-methyladenosine), m²A (2-methyladenosine), Am(2′-O-methyladenosine), ms²m⁶A (2-methylthio-N⁶-methyladenosine), I⁶A (N⁶-isopentenyladenosine), ms²i⁶A (2-methylthio-N6-isopentenyladenosine), io⁶A (N⁶-(cis-hydroxyisopentenyl)adenosine), ms²io⁶A (2-methylthio-N⁶-(cis-hydroxyisopentenyl)adenosine), G⁶A (N⁶-glycidylaminoformyladenosine), t⁶A (N⁶-threonylcarbamoyladenosine), ms²t⁶A (2-methylthio-N⁶-threonylcarbamoyladenosine), m⁶t⁶A (N⁶-methyl-N⁶-threonylcarbamoyladenosine), hn⁶A (N⁶-hydroxydemethylcarbamoyladenosine), ms²hn⁶A (2-methylthio-N⁶-hydroxyvalyl carbamoyladenosine). Ar(p) (2′-O-ribosyladenosine(phosphate)), I (inosine), m1I (1-methylinosine), m¹Im (1,2′-O-dimethylinosine), m³C (3-methylcytidine), Cm (2′-O-methylcytidine), s²C (2-thiocytidine), ac⁴C (N⁴-acetylcytidine), f⁵C (5-formylcytidine), m⁵Cm (-5,2′-O-dimethylcytidine), ac⁴Cm (N4-acetyl-2′-O-methylcytidine), k²C (lysidine), m¹G (1-methylguanosine), m²G (N²,N²-methylguanosine), m⁷G (7-methylguanosine), Gm (2′-O-methylguanosine), m²₂G (N²,N²-dimethylguanosine), m²Gm (N²,2′-O-dimethylguanosine), m²₂Gm (N²,N²,2′-O-trimethylguanosine), Gr(p) (2′-O-ribosylguanosine(phosphate)), yW (wybutosine), o₂yW (peroxywybutosine), OHyW (hydroxywybutosine), OHyW* (undermodified hydroxywybutosine), imG (wyosine), mimG (methylwyosine), Q (queuosine), oQ (epoxyqueuosine), galQ (galactosyl-queuosine), manQ (mannosyl-queuosine), preQo (7-cyano-7-deazaguanosine), preQi (7-aminomethyl-7-deazaguanosine), G⁺ (archaeosine), D (dihydrouridine), m⁵Um (5,2′-O-dimethyluridine), s⁴U (4-thiouridine), m⁵s²U (5-methyl-2-thiouridine), s²Um (2-thio-2′-O-methyluridine), acp³U (3-(3-amino-3-carboxypropyl)uridine), ho⁵U (5-hydroxyuridine), mo⁵U (5-methyluridine), cmo⁵U (uridine 5-hydroxyacetic acid), mcmo⁵U (uridine 5-methyl hydroxyacetate), chm⁵U (5-(carboxyhydroxymethyl)uridine)), mchm⁵U (5-(carboxyhydroxymethyl)uridine methyl ester), mcm⁵U (5-methoxycarbonylmethyluridine), mcm⁵Um (5-methoxycarbonylmethyl-2′-O-methyluridine), mcm⁵s²U (5-methoxycarbonylmethyl-2-thiouridine), nm⁵S²U (5-aminomethyl-2-thiouridine), mnm⁵U (5-methylaminomethyluridine), mnm⁵s²U (5-methylaminomethyl-2-thiouridine), mnm⁵se²U (5-methylaminomethyl-2-selenouridine), ncm⁵U (5-carbamoylmethyluridine), ncm⁵Um (5-carbamoylmethyl-2′-O-methyluridine), cmnm⁵U (5-carboxymethylaminomethyluridine), cmnm⁵Um (5-carboxymethylaminomethyl-2′-O-methyluridine), cmnm5s2U (5-carboxymethylaminomethyl thiouridine), m⁶²A (N⁶,N⁶-dimethyladenosine), Im (2′-O-methylinosine), m⁴C (N⁴-methylcytidine), m⁴Cm (N⁴,2′-O-dimethylcytidine), hm⁵C (5-hydroxymethylcytidine), m³U (3-methyluridine), cm⁵U (5-carboxymethyluridine), m⁶Am (N⁶,2′-O-dimethyladenosine), m⁶₂Am (N⁶,N⁶,O-2′-trimethyladenosine), m^{2,7}G (N²,7-dimethylguanosine), m2′2′7G (N²,N²,O-2′-trimethylguanosine), m³Um (-3,2′-O-dimethyluridine), M⁵D (5-methyldihydrouridine), f⁵Cm (5-formyl-2′-O-methylcytidine), m¹Gm (1,2′-O-dimethylguanosine), m¹Am (1,2′-O-dimethyladenosine), τm⁵U (5-taurinomethyluridine), τm⁵s²U (5-taurinomethyl-2-thiouridine)), imG-14 (4-demethylcytosine), imG2 (isoserine), ac⁶A (N⁶-acetyladenosine), or any combination thereof. Additional modified nucleosides can be found from Modomics (http://modomics.genesilico.pl/). Also see, U.S. Pat. No. 8,278,036, for a discussion of modified nucleosides and their incorporation into mRNA.

Modified Nucleotides

The modified nucleosides (e.g., the compound of Formula (I-a)) and nucleotides (e.g., the compound of Formula (I-e) or (I-g)) disclosed herein can be prepared from readily available starting materials using the following general methods and procedures. It should be understood that typical or preferred process conditions (i.e., reaction temperatures, times, mole ratios of reactants, solvents, pressures, etc.) are given; other process conditions can also be used unless otherwise stated. Optimum reaction conditions may vary with the particular reactants or solvent used, but such conditions can be determined by one skilled in the art by routine optimization procedures.

Preparation of modified nucleosides and nucleotides may involve the protection and deprotection of various chemical groups. The need for protection and deprotection, and the selection of appropriate protecting groups may be readily determined by one skilled in the art. The chemistry of protecting groups can be found, for example, in Greene, et al., Protective Groups in Organic Synthesis, 2d. Ed., Wiley & Sons, 1991, which is incorporated herein by reference in its entirety.

The reactions of the processes described herein can be carried out in suitable solvents, which can be readily selected by one skilled in the art of organic synthesis. Suitable solvents can be substantially nonreactive with the starting materials (reactants), the intermediates, or products at the temperatures at which the reactions are carried out, i.e., temperatures which can range from the solvent's freezing temperature to the solvent's boiling temperature. A given reaction may be carried out in one solvent or a mixture of more than one solvent. Depending on the particular reaction step, suitable solvents for a particular reaction step may be selected. Resolution of racemic mixtures of modified nucleosides and nucleotides may be carried out by any of numerous methods known in the art. An exemplary method includes fractional recrystallization using a “chiral resolving acid” which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids. Resolution of racemic mixtures can also be carried out by elution on a column packed with an optically active resolving agent (e.g., dinitrobenzoylphenylglycine). Suitable elution solvent composition may be determined by one skilled in the art.

Modified nucleosides and nucleotides may be prepared according to the synthesis scheme provided below:

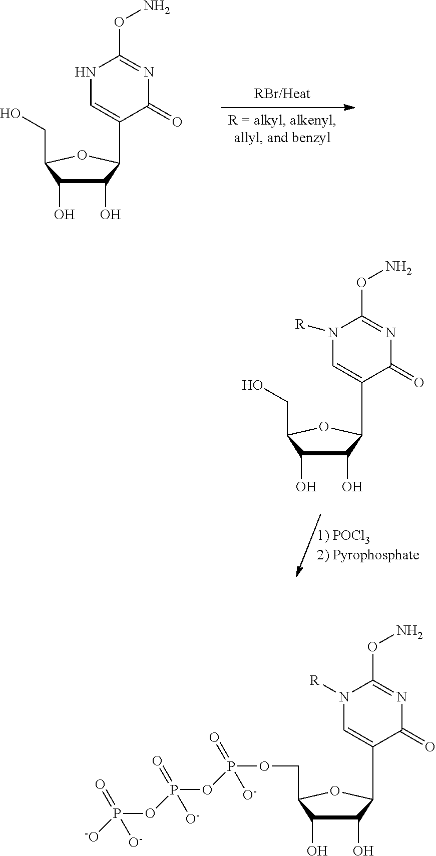

Modified nucleosides and nucleotides may be prepared according to the synthesis scheme provided below:

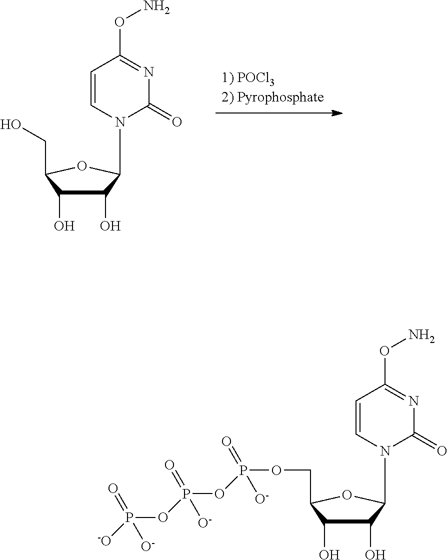

Modified nucleosides and nucleotides may also be prepared according to the synthetic methods described in Ogata et al. Journal of Organic Chemistry 74:2585-2588, 2009; Purmal et al. Nucleic Acids Research 22(1): 72-78, 1994; Fukuhara et al. Biochemistry 1(4): 563-568, 1962; and Xu et al. Tetrahedron 48(9): 1729-1740, 1992, each of which is incorporated by reference in its entirety.

Modified Nucleic Acids

Disclosed herein are a modified nucleic acid, for example, mRNA, and a method for synthesizing the same.

Nucleic acids used in accordance with the present disclosure may be prepared according to any prior art including, but not limited to chemical synthesis, enzymatic synthesis, which is generally termed in vitro transcription, enzymatic or chemical cleavage of a longer precursor, etc. Methods for synthesizing RNAs are known in the art (see, e.g., Gait, M. J.(ed.) Oligonucleotidesynthesis: a practical approach, Oxford [Oxfordshire], Washington, D.C.: IRL Press, 1984; and Herdewijn, P. (ed.) Oligonucleotide synthesis:methods and applications, Methods in Molecular Biology, v. 288 (Clifton, N.J.) Totowa, N.J.: Humana Press, 2005; both of which are incorporated herein by reference in their entirety). The mRNA may be produced with a reaction mixture including a RNA polymerase, a linear DNA template, and an RNA polymerase reaction buffer (e.g., nucleotides, for example, ribonucleotides). The use of RNA has been respectively disclosed in US Patent Publication US20120195936 and international publication WO2011012316, both of which are incorporated herein by reference in their entirety.

The RNA polymerase reaction buffer typically includes a salt/buffering agent, e.g., Tris, HEPES, ammonium sulfate, sodium bicarbonate, sodium citrate, sodium acetate, potassium phosphate sodium phosphate, sodium chloride, and magnesium chloride. The pH of the reaction mixture may be between about 6 to 8.5, from 6.5 to 8.0, from 7.0 to 7.5, and in some examples, the pH is 7.5.

In one example, the reaction mixture includes NTPs at a concentration ranging from 1-10 mM, DNA template at a concentration ranging from 0.01-0.5 mg/ml, and RNA polymerase at a concentration ranging from 0.01-0.1 mg/ml, e.g., the reaction mixture comprises NTPs at a concentration of 5 mM, the DNA template at a concentration of 0.1 mg/ml, and the RNA polymerase at a concentration of 0.05 mg/ml.

Naturally occurring or modified nucleosides and/or nucleotides can be used to prepare modified nucleic acids, for example, modified mRNA, according to the present disclosure. For example, a modified mRNA can comprise one or more natural nucleosides (e.g., adenosine, guanosine, cytidine, uridine); modified nucleosides (e.g., 2-aminoadenosine, 2-thiothymidine, inosine, pyrrolo-pyrimidine, 3-methyl adenosine, 5-methylcytidine, C-5 propynyl-cytidine, C-5 propynyl-pyridine, 2-aminoadenosine, C5-bromouridine, C5-fluorouridine, C5-iodouridine, C5-propynyl-pyridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, pseudouridine, (e.g., N-1-methyl-pseudouridine), 2-thiouridine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); modified phosphate groups (e.g., phosphorothioates and 5′-N-phosphoramidite linkages), or any combination thereof.

The RNA molecule (e.g., mRNA) may comprise at least two nucleotides. The nucleotides may be naturally occurring nucleotides or modified nucleotides. In some examples, the RNA molecule comprises about 5 nucleotides to about 5,000 nucleotides. In some examples, the RNA molecule comprises at least about 5 nucleotides. In some examples, the RNA molecule comprises at most about 5,000 nucleotides. In some examples, the RNA molecule comprises about 5 nucleotides to about 20 nucleotides, about 5 nucleotides to about 40 nucleotides, about 5 nucleotides to about 60 nucleotides, about 5 nucleotides to about 80 nucleotides, about 5 nucleotides to about 100 nucleotides, about 5 nucleotides to about 200 nucleotides, about 5 nucleotides to about 500 nucleotides, about 5 nucleotides to about 1,000 nucleotides, about 5 nucleotides to about 2,000 nucleotides, about 5 nucleotides to about 5,000 nucleotides, about 20 nucleotides to about 40 nucleotides, about 20 nucleotides to about 60 nucleotides, about 20 nucleotides to about 80 nucleotides, about 20 nucleotides to about 100 nucleotides, about 20 nucleotides to about 200 nucleotides, about 20 nucleotides to about 500 nucleotides, about 20 nucleotides to about 1,000 nucleotides, about 20 nucleotides to about 2,000 nucleotides, about 20 nucleotides to about 5,000 nucleotides, about 40 nucleotides to about 60 nucleotides, about 40 nucleotides to about 80 nucleotides, about 40 nucleotides to about 100 nucleotides, about 40 nucleotides to about 200 nucleotides, about 40 nucleotides to about 500 nucleotides, about 40 nucleotides to about 1,000 nucleotides, about 40 nucleotides to about 2,000 nucleotides, about 40 nucleotides to about 5,000 nucleotides, about 60 nucleotides to about 80 nucleotides, about 60 nucleotides to about 100 nucleotides, about 60 nucleotides to about 200 nucleotides, about 60 nucleotides to about 500 nucleotides, about 60 nucleotides to about 1,000 nucleotides, about 60 nucleotides to about 2,000 nucleotides, about 60 nucleotides to about 5,000 nucleotides, about 80 nucleotides to about 100 nucleotides, about 80 nucleotides to about 200 nucleotides, about 80 nucleotides to about 500 nucleotides, about 80 nucleotides to about 1,000 nucleotides, about 80 nucleotides to about 2,000 nucleotides, about 80 nucleotides to about 5,000 nucleotides, about 100 nucleotides to about 200 nucleotides, about 100 nucleotides to about 500 nucleotides, about 100 nucleotides to about 1,000 nucleotides, about 100 nucleotides to about 2,000 nucleotides, about 100 nucleotides to about 5,000 nucleotides, about 200 nucleotides to about 500 nucleotides, about 200 nucleotides to about 1,000 nucleotides, about 200 nucleotides to about 2,000 nucleotides, about 200 nucleotides to about 5,000 nucleotides, about 500 nucleotides to about 1,000 nucleotides, about 500 nucleotides to about 2,000 nucleotides, about 500 nucleotides to about 5,000 nucleotides, about 1,000 nucleotides to about 2,000 nucleotides, about 1,000 nucleotides to about 5,000 nucleotides, or about 2,000 nucleotides to about 5,000 nucleotides. In some examples, the RNA molecule comprises about 5 nucleotides, about 20 nucleotides, about 40 nucleotides, about 60 nucleotides, about 80 nucleotides, about 100 nucleotides, about 200 nucleotides, about 500 nucleotides, about 1,000 nucleotides, about 2,000 nucleotides, or about 5,000 nucleotides.

The RNA molecule (e.g., mRNA) may comprise at least one modified nucleotides described herein. In some examples, the RNA molecule comprises about 1 modified nucleotide to about 100 modified nucleotides. In some examples, the RNA molecule comprises at least about 1 modified nucleotide. In some examples, the RNA molecule comprises at most about 100 modified nucleotides. In some examples, the RNA molecule comprises about 1 modified nucleotide to about 2 modified nucleotides, about 1 modified nucleotide to about 3 modified nucleotides, about 1 modified nucleotide to about 4 modified nucleotides, about 1 modified nucleotide to about 5 modified nucleotides, about 1 modified nucleotide to about 10 modified nucleotides, about 1 modified nucleotide to about 20 modified nucleotides, about 1 modified nucleotide to about 100 modified nucleotides, about 2 modified nucleotides to about 3 modified nucleotides, about 2 modified nucleotides to about 4 modified nucleotides, about 2 modified nucleotides to about 5 modified nucleotides, about 2 modified nucleotides to about 10 modified nucleotides, about 2 modified nucleotides to about 20 modified nucleotides, about 2 modified nucleotides to about 100 modified nucleotides, about 3 modified nucleotides to about 4 modified nucleotides, about 3 modified nucleotides to about 5 modified nucleotides, about 3 modified nucleotides to about 10 modified nucleotides, about 3 modified nucleotides to about 20 modified nucleotides, about 3 modified nucleotides to about 100 modified nucleotides, about 4 modified nucleotides to about 5 modified nucleotides, about 4 modified nucleotides to about 10 modified nucleotides, about 4 modified nucleotides to about 20 modified nucleotides, about 4 modified nucleotides to about 100 modified nucleotides, about 5 modified nucleotides to about 10 modified nucleotides, about 5 modified nucleotides to about 20 modified nucleotides, about 5 modified nucleotides to about 100 modified nucleotides, about 10 modified nucleotides to about 20 modified nucleotides, about 10 modified nucleotides to about 100 modified nucleotides, or about 20 modified nucleotides to about 100 modified nucleotides. In some examples, the RNA molecule comprises about 1 modified nucleotide, about 2 modified nucleotides, about 3 modified nucleotides, about 4 modified nucleotides, about 5 modified nucleotides, about 10 modified nucleotides, about 20 modified nucleotides, or about 100 modified nucleotides.

The RNA molecule (e.g., mRNA) may comprise at least 0.1% modified nucleotides. The fraction of modified nucleotides can be calculated as: number of modified nucleotides/total number of nucleotides * 100%. In some examples, the RNA molecule comprises about 0.1% modified nucleotides to about 100% modified nucleotides. In some examples, the RNA molecule comprises at least about 0.1% modified nucleotides. In some examples, the RNA molecule comprises at most about 100% modified nucleotides. In some examples, the RNA molecule comprises about 0.1% modified nucleotides to about 0.2% modified nucleotides, about 0.1% modified nucleotides to about 0.5% modified nucleotides, about 0.1% modified nucleotides to about 1% modified nucleotide, about 0.1% modified nucleotides to about 2% modified nucleotides, about 0.1% modified nucleotides to about 5% modified nucleotides, about 0.1% modified nucleotides to about 10% modified nucleotides, about 0.1% modified nucleotides to about 20% modified nucleotides, about 0.1% modified nucleotides to about 50% modified nucleotides, about 0.1% modified nucleotides to about 100% modified nucleotides, about 0.2% modified nucleotides to about 0.5% modified nucleotides, about 0.2% modified nucleotides to about 1% modified nucleotide, about 0.2% modified nucleotides to about 2% modified nucleotides, about 0.2% modified nucleotides to about 5% modified nucleotides, about 0.2% modified nucleotides to about 10% modified nucleotides, about 0.2% modified nucleotides to about 20% modified nucleotides, about 0.2% modified nucleotides to about 50% modified nucleotides, about 0.2% modified nucleotides to about 100% modified nucleotides, about 0.5%modified nucleotides to about 1% modified nucleotide, about 0.5% modified nucleotides to about 2% modified nucleotides, about 0.5% modified nucleotides to about 5% modified nucleotides, about 0.5% modified nucleotides to about 10% modifiednucleotides, about 0.5% modified nucleotides to about 20% modified nucleotides, about 0.5% modified nucleotides to about 50% modified nucleotides, about 0.5% modified nucleotides to about 100% modified nucleotides, about 1% modified nucleotide to about 2% modified nucleotides, about 1% modified nucleotide to about 5% modified nucleotides, about 1% modified nucleotide to about 10% modified nucleotides, about 1% modified nucleotide to about 20% modified nucleotides, about 1% modified nucleotide to about 50% modified nucleotides, about 1% modified nucleotide to about 100% modified nucleotides, about 2% modified nucleotides to about 5% modified nucleotides, about 2% modified nucleotides to about 10% modified nucleotides, about 2% modified nucleotides to about 20% modified nucleotides, about 2% modified nucleotides to about 50% modified nucleotides, about 2% modified nucleotides to about 100% modified nucleotides, about 5% modified nucleotides to about 10% modified nucleotides, about 5% modified nucleotides to about 20% modified nucleotides, about 5% modified nucleotides to about 50% modified nucleotides, about 5% modified nucleotides to about 100% modified nucleotides, about 10% modified nucleotides to about 20% modified nucleotides, about 10% modified nucleotides to about 50% modified nucleotides, about 10% modified nucleotides to about 100% modified nucleotides, about 20% modified nucleotides to about 50% modified nucleotides, about 20% modified nucleotides to about 100% modified nucleotides, or about 50% modified nucleotides to about 100% modified nucleotides. In some examples, the RNA molecule comprises about 0.1% modified nucleotides, about 0.2% modified nucleotides, about 0.5% modified nucleotides, about 1% modified nucleotide, about 2% modified nucleotides, about 5% modified nucleotides, about 10% modified nucleotides, about 20% modified nucleotides, about 50% modified nucleotides, or about 100% modified nucleotides.

In some examples, a compound of Formula (I) or (I-a) replaces about 1 nucleoside (e.g., uridine or cytidine) in the modified RNA to about 10,000 nucleosides (e.g., uridine or cytidine) in the modified RNA. In some examples, a compound of Formula (I) or (I-a) replaces at least about 1 nucleoside in the modified RNA. In some examples, a compound of Formula (I) or (I-a) replaces at most about 10,000 nucleosides in the modified RNA. In some examples, a compound of Formula (I) or (I-a) replaces about 1 nucleoside in the modified RNA to about 2 nucleosides in the modified RNA, about 1 nucleoside in the modified RNA to about 10 nucleosides in the modified RNA, about 1 nucleoside in the modified RNA to about 50 nucleosides in the modified RNA, about 1 nucleoside in the modified RNA to about 100 nucleosides in the modified RNA, about 1 nucleoside in the modified RNA to about 500 nucleosides in the modified RNA, about 1 nucleoside in the modified RNA to about 1,000 nucleosides in the modified RNA, about 1 nucleoside in the modified RNA to about 5,000 nucleosides in the modified RNA, about 1 nucleoside in the modified RNA to about 10,000 nucleosides in the modified RNA, about 2 nucleosides in the modified RNA to about 10 nucleosides in the modified RNA, about 2 nucleosides in the modified RNA to about 50 nucleosides in the modified RNA, about 2 nucleosides in the modified RNA to about 100 nucleosides in the modified RNA, about 2 nucleosides in the modified RNA to about 500 nucleosides in the modified RNA, about 2 nucleosides in the modified RNA to about 1,000 nucleosides in the modified RNA, about 2 nucleosides in the modified RNA to about 5,000 nucleosides in the modified RNA, about 2 nucleosides in the modified RNA to about 10,000 nucleosides in the modified RNA, about 10 nucleosides in the modified RNA to about 50 nucleosides in the modified RNA, about 10 nucleosides in the modified RNA to about 100 nucleosides in the modified RNA, about 10 nucleosides in the modified RNA to about 500 nucleosides in the modified RNA, about 10 nucleosides in the modified RNA to about 1,000 nucleosides in the modified RNA, about 10 nucleosides in the modified RNA to about 5,000 nucleosides in the modified RNA, about 10 nucleosides in the modified RNA to about 10,000 nucleosides in the modified RNA, about 50 nucleosides in the modified RNA to about 100 nucleosides in the modified RNA, about 50 nucleosides in the modified RNA to about 500 nucleosides in the modified RNA, about 50 nucleosides in the modified RNA to about 1,000 nucleosides in the modified RNA, about 50 nucleosides in the modified RNA to about 5,000 nucleosides in the modified RNA, about 50 nucleosides in the modified RNA to about 10,000 nucleosides in the modified RNA, about 100 nucleosides in the modified RNA to about 500 nucleosides in the modified RNA, about 100 nucleosides in the modified RNA to about 1,000 nucleosides in the modified RNA, about 100 nucleosides in the modified RNA to about 5,000 nucleosides in the modified RNA, about 100 nucleosides in the modified RNA to about 10,000 nucleosides in the modified RNA, about 500 nucleosides in the modified RNA to about 1,000 nucleosides in the modified RNA, about 500 nucleosides in the modified RNA to about 5,000 nucleosides in the modified RNA, about 500 nucleosides in the modified RNA to about 10,000 nucleosides in the modified RNA, about 1,000 nucleosides in the modified RNA to about 5,000 nucleosides in the modified RNA, about 1,000 nucleosides in the modified RNA to about 10,000 nucleosides in the modified RNA, or about 5,000 nucleosides in the modified RNA to about 10,000 nucleosides in the modified RNA. In some examples, a compound of Formula (I) or (I-a) replaces about 1 nucleoside in the modified RNA, about 2 nucleosides in the modified RNA, about 10 nucleosides in the modified RNA, about 50 nucleosides in the modified RNA, about 100 nucleosides in the modified RNA, about 500 nucleosides in the modified RNA, about 1,000 nucleosides in the modified RNA, about 5,000 nucleosides in the modified RNA, or about 10,000 nucleosides in the modified RNA.

In some examples, a compound of Formula (I) or (I-a) replaces about 0.01% of the nucleosides (e.g., uridine or cytidine) in the modified RNA to about 100% of the nucleosides (e.g., uridine or cytidine) in the modified RNA. In some examples, a compound of Formula (I) or (I-a) replaces at least about 0.01% of the nucleosides in the modified RNA. In some examples, a compound of Formula (I) or (I-a) replaces at most about 100% of the nucleosides in the modified RNA. In some examples, a compound of Formula (I) or (I-a) replaces about 0.01% of the nucleosides in the modified RNA to about 0.1% of the nucleosides in the modified RNA, about 0.01% of the nucleosides in the modified RNA to about 0.5% of the nucleosides in the modified RNA, about 0.01% of the nucleosides in the modified RNA to about1% of the nucleosides in the modified RNA, about 0.01% of the nucleosides in the modified RNA to about 5% of the nucleosides in the modified RNA, about 0.01% of the nucleosides in the modified RNA to about 10% of the nucleosides in the modified RNA, about 0.01% of the nucleosides in the modified RNA to about 50% of the nucleosides in the modified RNA, about 0.01% of the nucleosides in the modified RNA to about 100% of the nucleosides in the modified RNA, about 0.1% of the nucleosides in the modified RNA to about 0.5% of the nucleosides in the modified RNA, about 0.1% of the nucleosides in the modified RNA to about 1% of the nucleosides in the modified RNA, about 0.1% of the nucleosides in the modified RNA to about 5% of the nucleosides in the modified RNA, about 0.1% of the nucleosides in the modified RNA to about 10% of the nucleosides in the modified RNA, about 0.1% of the nucleosides in the modified RNA to about 50% of the nucleosides in the modified RNA, about 0.1% of the nucleosides in the modified RNA to about 100% of the nucleosides in the modified RNA, about 0.5% of the nucleosides in the modified RNA to about 1% of the nucleosides in the modified RNA, about 0.5% of the nucleosides in the modified RNA to about 5% of the nucleosides in the modified RNA, about 0.5% of the nucleosides in the modified RNA to about 10% of the nucleosides in the modified RNA, about 0.5% of the nucleosides in the modified RNA to about 50% of the nucleosides in the modified RNA, about 0.5% of the nucleosides in the modified RNA to about 100% of the nucleosides in the modified RNA, about 1% of the nucleosides in the modified RNA to about 5% of the nucleosides in the modified RNA, about 1% of the nucleosides in the modified RNA to about 10% of the nucleosides in the modified RNA, about 1% of the nucleosides in the modified RNA to about 50% of the nucleosides in the modified RNA, about 1% of the nucleosides in the modified RNA to about 100% of the nucleosides in the modified RNA, about 5% of the nucleosides in the modified RNA to about 10% of the nucleosides in the modified RNA, about 5% of the nucleosides in the modified RNA to about 50% of the nucleosides in the modified RNA, about 5% of the nucleosides in the modified RNA to about 100% of the nucleosides in the modified RNA, about 10% of the nucleosides in the modified RNA to about 50% of the nucleosides in the modified RNA, about 10% of the nucleosides in the modified RNA to about 100% of the nucleosides in the modified RNA, or about 50% of the nucleosides in the modified RNA to about 100% of the nucleosides in the modified RNA. In some examples, a compound of Formula (I) or (I-a) replaces about 0.01% of the nucleosides in the modified RNA, about 0.1% of the nucleosides in the modified RNA, about 0.5% of the nucleosides in the modified RNA, about 1% of the nucleosides in the modified RNA, about 5% of the nucleosides in the modified RNA, about 10% of the nucleosides in the modified RNA, about 50% of the nucleosides in the modified RNA, or about 100% of the nucleosides in the modified RNA.

The concentration of each nucleotide, such as ribonucleotide (e.g., ATP, UTP, GTP, and CTP), in the reaction mixture may be between about 0.1 mM and about 100 mM. In some examples, the concentration of each nucleotide is at least about 0.1 mM. In some examples, the concentration of each nucleotide is at most about 100 mM. In some examples, the concentration of each nucleotide is about 0.1 mM to about 0.5 mM, about 0.1 mM to about 1 mM, about 0.1 mM to about 5 mM, about 0.1 mM to about 10 mM, about 0.1 mM to about 20 mM, about 0.1 mM to about 50 mM, about 0.1 mM to about 75 mM, about 0.1 mM to about 100 mM, about 0.5 mM to about 1 mM, about 0.5 mM to about 5 mM, about 0.5 mM to about 10 mM, about 0.5 mM to about 20 mM, about 0.5 mM to about 50 mM, about 0.5 mM to about 75 mM, about 0.5 mM to about 100 mM, about 1 mM to about 5 mM, about 1 mM to about 10 mM, about 1 mM to about 20 mM, about 1 mM to about 50 mM, about 1 mM to about 75 mM, about 1 mM to about 100 mM, about 5 mM to about 10 mM, about 5 mM to about 20 mM, about 5 mM to about 50 mM, about 5 mM to about 75 mM, about 5 mM to about 100 mM, about 10 mM to about 20 mM, about 10 mM to about 50 mM, about 10 mM to about 75 mM, about 10 mM to about 100 mM, about 20 mM to about 50 mM, about 20 mM to about 75 mM, about 20 mM to about 100 mM, about 50 mM to about 75 mM, about 50 mM to about 100 mM, or about 75 mM to about 100 mM. In some examples, the concentration of each nucleotide is about 0.1 mM, about 0.5 mM, about 1 mM, about 5 mM, about 10 mM, about 20 mM, about 50 mM, about 75 mM, or about 100 mM.

The total concentration of nucleotides, (e.g., ATP, GTP, CTP and UTP combined), used in the reaction ranges between 0.5 mM to about 500 mM. In some examples, the total concentration of nucleotides is about 0.5 mM to about 500 mM. In some examples, the total concentration of nucleotides is at least about 0.5 mM. In some examples, the concentration of each nucleotide is at most about 500 mM. In some examples, the total concentration of nucleotides is about 0.5 mM to about 1 mM, about 0.5 mM to about 5 mM, about 0.5 mM to about 10 mM, about 0.5 mM to about 50 mM, about 0.5 mM to about 100 mM, about 0.5 mM to about 200 mM, about 0.5 mM to about 300 mM, about 0.5 mM to about 500 mM, about 1 mM to about 5 mM, about 1 mM to about 10 mM, about 1 mM to about 50 mM, about 1 mM to about 100 mM, about 1 mM to about 200 mM, about 1 mM to about 300 mM, about 1 mM to about 500 mM, about 5 mM to about 10 mM, about 5 mM to about 50 mM, about 5 mM to about 100 mM, about 5 mM to about 200 mM, about 5 mM to about 300 mM, about 5 mM to about 500 mM, about 10 mM to about 50 mM, about 10 mM to about 100 mM, about 10 mM to about 200 mM, about 10 mM to about 300 mM, about 10 mM to about 500 mM, about 50 mM to about 100 mM, about 50 mM to about 200 mM, about 50 mM to about 300 mM, about 50 mM to about 500 mM, about 100 mM to about 200 mM, about 100 mM to about 300 mM, about 100 mM to about 500 mM, about 200 mM to about 300 mM, about 200 mM to about 500 mM, or about 300 mM to about 500 mM. In some examples, the total concentration of nucleotides is about 0.5 mM, about 1 mM, about 5 mM, about 10 mM, about 50 mM, about 100 mM, about 200 mM, about 300 mM, or about 500 mM.

Post-Synthesis Processing

A 5′ cap and/or a 3′ tail may be added after the synthesis. The presence of the cap may provide resistance to nucleases found in most eukaryotic cells. The presence of a “tail” may serve to protect the mRNA from exonuclease degradation and/or modulate the protein expression level.

A 5′ cap may be added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5)ppp (5′(A,G(5′)ppp(5′)A and G(5′)ppp(5′)G. More cap structures have been described in published US Application No. US 2016/0032356, Ashiqul Haque et al., “Chemically modified hCFTR mRNAs recuperate lung function in a mouse model of cystic fibrosis,” Scientific Reports (2018) 8:16776, and Kore et al., “Recent Developments in 5′-Terminal Cap Analogs:Synthesis and Biological Ramifications,” Mini-Reviews in Organic Chemistry, 2008, 5, 179-192, which are incorporated herein by reference.

A tail structure may include a poly(A) and/or poly(C) tail. A poly-A tail on the 3′ terminus (e.g. 10, 20, 30, 40, 50, 60, 70, 80, 90, or 100 nucleotides on the 3′ terminus) of mRNA may include at least 50%, 55%, 65%, 70%, 75%, 80%, 85%, 90%, 92%, 94%, 95%, 96%, 97%, 98%, or 99% adenosine nucleotides. A poly-A tail on the 3′ terminus (e.g. 10, 20, 30, 40, 50, 60, 70, 80, 90, or 100 nucleotides on the 3′ terminus) of mRNA can include at least 50%, 55%, 65%, 70%, 75%, 80%, 85%, 90%, 92%, 94%, 95%, 96%, 97%, 98%, or 99% cytosine nucleotides or uracil nucleotides.

As described herein, the addition of the 5′ cap and/or the 3′ tail may facilitate the detection of abortive transcripts generated during in vitro synthesis, because without capping and/or tailing, the size of those prematurely aborted mRNA transcripts can be too small to be detected. Thus, in some examples, the 5′ cap and/or the 3′ tail are added to the synthesized mRNA before the mRNA is tested for purity (e.g., the level of abortive transcripts present in the mRNA). In some examples, the 5′ cap and/or the 3′ tail are added to the synthesized mRNA before the mRNA is purified as described herein. In other examples, the 5′ cap and/or the 3′ tail are added to the synthesized mRNA after the mRNA is purified as described herein.

mRNA synthesized according to the present disclosure may be used without further purification. In particular, mRNA synthesized according to the present disclosure may be used without a step of removing shortmers. In some examples, mRNA synthesized according to the present disclosure may be further purified. Various methods may be used to purify mRNA synthesized according to the present disclosure. For example, purification of mRNA may be performed using centrifugation, filtration and/or chromatographic methods. In some examples, the synthesized mRNA is purified by ethanol precipitation or filtration or chromatography, or gel purification or any other suitable means. In some examples, the mRNA is purified by HPLC. In some examples, the mRNA is extracted in a standard phenol: chloroform: isoamyl alcohol solution, which is well known to one skilled in the art. In some examples, the mRNA is purified using Tangential Flow Filtration. Suitable purification methods include those described in US 2016/0040154, US 2015/0376220, PCT application PCT/US18/19954 entitled “METHODS FOR PURIFICATION OF DIGESTANT RNA” filed on Feb. 27, 2018, and PCT application PCT/US18/19978 entitled “METHODS FOR PURIFICATION OF MESSENGER RNA” filed on Feb. 27, 2018, all of which are incorporated by reference herein and may be used to practice the present disclosure.

In some examples, the mRNA is purified before capping and tailing. In some examples, the mRNA is purified after capping and tailing. In some examples, the mRNA is purified both before and after capping and tailing. In some examples, the mRNA is purified either before or after or both before and after capping and tailing, by centrifugation. In some examples, the mRNA is purified either before or after or both before and after capping and tailing, by filtration. In some examples, the mRNA is purified either before or after or both before and after capping and tailing, by Tangential Flow Filtration (TFF). In some examples, the mRNA is purified either before or after or both before and after capping and tailing by chromatography.

Full-length or abortive transcripts of mRNA can be detected and quantified using any methods available in the art. In some examples, the synthesized mRNA molecules are detected using blotting, capillary electrophoresis, chromatography, fluorescence, gel electrophoresis, HPLC, silver stain, spectroscopy, ultraviolet (UV), or Ultra Performance Liquid Chromatography (UPLC), or a combination thereof. Other detection methods known in the art are included in the present disclosure. In some examples, the synthesized mRNA molecules are detected using UV absorption spectroscopy with separation by capillary electrophoresis. In some examples, mRNA is first denatured by a Glyoxal dye before gel electrophoresis (“Glyoxal Gel Electrophoresis”). In some examples, synthesized mRNA is characterized before capping or tailing. In some examples, synthesized mRNA is characterized after capping and tailing.

In some examples, mRNA generated by the method disclosed herein comprises less than 10%, less than 9%, less than 8%, less than 7%, less than 6%, less than 5%, less than 4%, less than 3%, less than 2%, less than 1%, less than 0.5%, less than 0.1% impurities other than the full-length mRNA. The impurities include IVT contaminants, e.g., proteins, enzymes, free nucleotides and/or shortmers.

In some examples, mRNA produced according to the disclosure is substantially free of shortmers or abortive transcripts. In particular, mRNA produced according to the disclosure contains undetectable level of shortmers or abortive transcripts by capillary electrophoresis or Glyoxal gel electrophoresis. As used herein, the term “shortmer” or “abortive transcript” refers to any transcript that is less than the full-length. In some examples, “shortmers” or “abortive transcripts” are less than 100 nucleotides in length, less than 90, less than 80, less than 70, less than 60, less than 50, less than 40, less than 30, less than 20, or less than 10 nucleotides inlength. In some examples, shortmers are detected or quantified after adding a 5′-cap, and/or a 3′-poly A tail.

Pharmaceutical Composition

Also provided are pharmaceutical compositions comprising compounds, modified nucleosides, modified nucleotides, or modified nucleic acids provided herein.

In some examples, the pharmaceutical compositions according to the present disclosure may be administered to a subject by any method known to a person skilled in the art, such as parenterally, orally, transmucosally, transdermally, intramuscularly, intravenously, intra-dermally, subcutaneously, intra-peritonealy, intra-ventricularly, intra-cranially, intra-vaginally or intra-tumorally.

The pharmaceutical compositions may be administered by intravenous, intra-arterial, or intra-muscular injection of a liquid preparation. Suitable liquid formulations include solutions, suspensions, dispersions, emulsions, oils and the like. In some examples, the pharmaceutical compositions are administered intravenously and are thus formulated in a form suitable for intravenous administration. In some examples, the pharmaceutical compositions are administered intra-arterially and are thus formulated in a form suitable for intra-arterial administration. In some examples, the pharmaceutical compositions are administered intra-muscularly and are thus formulated in a form suitable for intra-muscular administration.

The pharmaceutical compositions can be delivered in a vesicle, e.g. a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid).

The pharmaceutical compositions may be administered orally, and may be thus formulated in a form suitable for oral administration, i.e. as a solid or a liquid preparation. Suitable solid oral formulations may include tablets, capsules, granules, pills and the like. Suitable liquid oral formulations may include solutions, suspensions, dispersions, emulsions, oils.

The pharmaceutical compositions may be administered topically to body surfaces and may be thus formulated in a form suitable for topical administration. Suitable topical formulations may include gels, ointments, creams, lotions, drops and the like. For topical administration, the compositions or their physiologically tolerated derivatives may be prepared and applied as solutions, suspensions, or emulsions in a physiologically acceptable diluent with or without a pharmaceutical carrier. The pharmaceutical compositions may be administered as a suppository, for example a rectal suppository or a urethral suppository. In some examples, the pharmaceutical composition is administered by subcutaneous implantation of a pellet. In some examples, the pellet provides for controlled release of agent over a period of time.

The pharmaceutical compositions may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21^{st} Edition, A. R. Gennaro (Lippincott, Williams & Wilkins, Baltimore, Md., 2006; incorporated herein by reference) discloses various excipients used in formulating pharmaceutical compositions and known techniques for the preparation thereof.

In some examples, a pharmaceutically acceptable excipient is at least 95%, at least 96%, at least 97%, at least 98%, at least 99%, or 100% pure. In some examples, an excipient is approved for use in humans and for veterinary use. In some examples, an excipient is approved by United States Food and Drug Administration. In some examples, an excipient is pharmaceutical grade. In some examples, an excipient meets the standards of the United States Pharmacopoeia (USP), the European Pharmacopoeia (EP), the British Pharmacopoeia, and/or the International Pharmacopoeia.

The pharmaceutically acceptable carriers for liquid formulations may be aqueous or non-aqueous solutions, suspensions, emulsions or oils. Examples of non-aqueous solvents may be propylene glycol, polyethylene glycol, and injectable organic esters such as ethyl oleate. Aqueous carriers may include water, alcoholic/aqueous solutions, emulsions or suspensions, including saline and buffered media. Examples of oils may be those of petroleum, animal, vegetable, or synthetic origin, for example, peanut oil, soybean oil, mineral oil, olive oil, sunflower oil, and fish-liver oil.

Parenteral vehicles (for subcutaneous, intravenous, intraarterial, or intramuscular injection) may include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples may be sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions. Examples of oils may be those of petroleum, animal, vegetable, or synthetic origin, for example, peanut oil, soybean oil, mineral oil, olive oil, sunflower oil, and fish-liver oil.

The pharmaceutical compositions can further comprise binders (e.g. acacia, cornstarch, gelatin, carbomer, ethyl cellulose, guar gum, hydroxypropyl cellulose, hydroxypropyl methyl cellulose, povidone), disintegrating agents (e.g. cornstarch, potato starch, alginic acid, silicon dioxide, croscarmelose sodium, crospovidone, guar gum, sodium starch glycolate), buffers (e.g., Tris-HCI., acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., glycerol, polyethylene glycerol), anti-oxidants (e.g., ascorbic acid, sodium metabisulfite, butylated hydroxyanisole), stabilizers (e.g. hydroxypropyl cellulose, hyroxypropylmethyl cellulose), viscosity increasing agents (e.g. carbomer, colloidal silicon dioxide, ethyl cellulose, guar gum), sweeteners (e.g. aspartame, citric acid), preservatives (e.g., Thimerosal, benzyl alcohol, parabens), lubricants (e.g. stearic acid, magnesium stearate, polyethylene glycol, sodium lauryl sulfate), flow-aids (e.g. colloidal silicon dioxide), plasticizers (e.g. diethyl phthalate, triethyl citrate), emulsifiers (e.g. carbomer, hydroxypropyl cellulose, sodium lauryl sulfate), polymer coatings (e.g., poloxamers or poloxamines), coating and film forming agents (e.g. ethyl cellulose, acrylates, polymethacrylates) and/or adjuvants.

The pharmaceutical compositions provided herein may be controlled-release compositions, i.e., compositions in which the compound is released over a period of time after administration. Controlled- or sustained-release compositions may include formulation in lipophilic depots (e.g. fatty acids, waxes, oils). In some examples, the pharmaceutical composition may be an immediate-release composition, i.e. a composition in which the entire compound is released immediately after administration.

Suitable devices for use in delivering intradermal pharmaceutical compositions described herein may include short needle devices such as those described in U.S. Pat. Nos. 4,886,499; 5,190,521; 5,328,483; 5,527,288; 4,270,537; 5,015,235; 5,141,496; and 5,417,662. Intradermal compositions can be administered by devices which limit the effective penetration length of a needle into the skin, such as those described in PCT publication WO 99/34850 and functional equivalents thereof. Jet injection devices which deliver liquid compositions to the dermis via a liquid jet injector and/or via a needle which pierces the stratum corneum and produces a jet which reaches the dermis may be suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537. Ballistic powder/particle delivery devices which use compressed gas to accelerate vaccine in powder form through the outer layers of the skin to the dermis can be suitable. Alternatively or additionally, conventional syringes may be used in the classical mantoux method of intradermal administration.

mRNA synthesized according to the present disclosure may be formulated and delivered for in vivo protein production using any method. In some examples, mRNA is encapsulated, into a transfer vehicle, such as a nanoparticle. Moreover, one purpose of such encapsulation is often to protect the nucleic acid from an environment which may contain enzymes or chemicals that degrade nucleic acids and/or systems or receptors that cause the rapid excretion of the nucleic acids. Accordingly, in some examples, a suitable delivery vehicle is capable of enhancing the stability of the mRNA contained therein and/or facilitate the delivery of mRNA to the target cell or tissue. In some examples, nanoparticles may be lipid-based nanoparticles, e.g., comprising a liposome, or polymer-based nanoparticles. In some examples, a nanoparticle may have a diameter of less than about 40-100 nm. A nanoparticle may include at least 1 μg, 10 μg, 100 μg, 1 mg, 10 mg, 100 mg, 1 g, or more mRNA.

In some examples, the transfer vehicle is a liposomal vesicle, or other means to facilitate the transfer of a nucleic acid to target cells and tissues. Suitable transfer vehicles can include, but are not limited to, liposomes, nanoliposomes, ceramide-containing nanoliposomes, proteoliposomes, nanoparticulates, calcium phosphor-silicate nanoparticulates, calcium phosphate nanoparticulates, silicon dioxide nanoparticulates, nanocrystalline particulates, semiconductor nanoparticulates, poly(D-arginine), nanodendrimers, starch-based delivery systems, micelles, emulsions, niosomes, plasmids, viruses, calcium phosphate nucleotides, aptamers, peptides and other vectorial tags. Also contemplated may be the use of bionanocapsules and other viral capsid proteins assemblies as a suitable transfer vehicle. (Hum. Gene Ther. 2008 September; 19(9):887-95).

A liposome may include one or more cationic lipids, one or more non-cationic lipids, one or more sterol-based lipids, and/or one or more PEG-modified lipids. A liposome may include three or more distinct components of lipids, one distinct component of lipids being sterol-based cationic lipids. In some examples, the sterol-based cationic lipid is an imidazole cholesterol ester or “ICE” lipid (see, WO 2011/068810, which is incorporated by reference in its entirety). In some examples, sterol-based cationic lipids can constitute no more than 70% (e.g., no more than 65% and 60%) of the total lipids in a lipid nanoparticle (e.g., liposome).

Examples of suitable lipids may include, for example, the phosphatidyl compounds (e.g., phosphatidylglycerol, phosphatidylcholine, phosphatidylserine, phosphatidylethanolamine, sphingolipids, cerebrosides, and gangliosides).

Non-limiting examples of cationic lipids may include C12-200, MC3, DLinDMA, DLinkC2DMA, cKK-E12, ICE (Imidazole-based), HGT5000, HGT5001, OF-02, DODAC, DDAB, DMRIE, DOSPA, DOGS, DODAP, DODMA and DMDMA, DODAC, DLenDMA, DMRIE, CLinDMA, CpLinDMA, DMOBA, DOcarbDAP, DLinDAP, DLincarbDAP, DLinCDAP, KLin-K-DMA, DLin-K-XTC2-DMA, and HGT4003, or a combination thereof.

Non-limiting examples of non-cationic lipids can include ceramide; cephalin; cerebrosides; diacylglycerols; 1,2-dipalmitoyl-sn-glycero-3-phosphorylglycerol sodium salt (DPPG); 1,2-distearoyl-sn-glycero-3-phosphoethanolamine (DSPE); 1,2-distearoyl-sn-glycero-3-phosphocholine (DSPC); 1,2-dipalmitoyl-sn-glycero-3-phosphocholine (DPPC); 1,2-dioleyl-sn-glycero-3-phosphoethanolamine (DOPE); 1,2-dioleyl-sn-glycero-3-phosphotidylcholine (DOPC); 1,2-dipalmitoyl-sn-glycero-3-phosphoethanolamine (DPPE); 1,2-dimyristoyl-sn-glycero-3-phosphoethanolamine (DMPE); and 1,2-dioleoyl-sn-glycero-3-phospho-(1′-rac-glycerol) (DOPG), 1-palmitoyl-2-oleoyl-phosphatidylethanolamine (POPE); 1-palmitoyl-2-oleoyl-sn-glycero-3-phosphocholine (POPC); 1-stearoyl-2-oleoyl-phosphatidylethanolamine (SOPE); sphingomyelin; or a combination thereof.

In some examples, a PEG-modified lipid may be a poly(ethylene) glycol chain of up to 5 kDa in length covalently attached to a lipid with alkyl chain(s) of C6-C20 length. Non-limiting examples of PEG-modified lipids may include DMG-PEG, DMG-PEG2K, C8-PEG, DOG PEG, ceramide PEG, and DSPE-PEG, or a combination thereof.

Also contemplated may be the use of polymers as transfer vehicles, whether alone or in combination with other transfer vehicles. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins and polyethylenimine. A polymer-based nanoparticles may include polyethylenimine (PEI), e.g., a branched PEI.

Although methods and materials similar or equivalent to those described herein can be used in the practice or testing of the present disclosure, suitable methods and materials are described below. All publications, patent applications, patents, and other references mentioned herein are incorporated by reference in their entirety. The references cited herein are not admitted to be prior art to the disclosure claimed. In addition, the materials, methods, and examples are illustrative only and are not intended to be limiting.

### DETAILED DESCRIPTION OF THE EMBODIMENTS

The novel features of the disclosure are set forth in the appended claims specifically. A better understanding of the features and advantages of the present disclosure will be obtained by reference to the following detailed description that sets forth illustrative embodiments, in which the principles of the disclosure are utilized, and the accompanying drawings of which: disclosed herein are compounds, modified nucleosides, modified nucleotides, modified nucleic acids, and methods for synthesizing the same.

### EXAMPLE 1

### Synthesis of 1-((2R,3R,4R,5R)-3,4-bis((tert-butyldimethylsilyl chloride(yl))oxy)-5-(((tert-butyldimethylsilyl chloride(yl))oxy)methyl)tetrahydrofuran pyridine-2-yl)pyrimidin-2,4(1H,3H)-dione)

The title compound was synthesized via the following reaction:

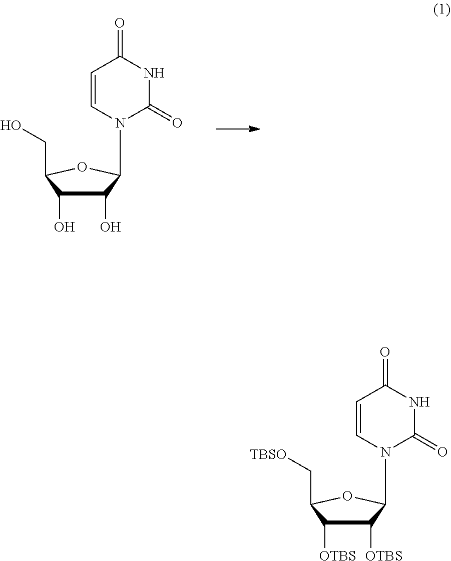

Uridine nucleoside (1.22 g, 5 mmol), imidazole (1.36 g, 20 mmol), 4-dimethylaminopyridine (DMAP) (0.31 g, 2.5 mmol), tert-butyldimethylsilyl chloride (3.02 g, 20 mmol), and N,N-dimethylformamide (DMF) (20 mL) were mixed together in a reaction flask and stirred overnight at 60° C. The reaction mixture was then poured into ice water (150 mL) and washed with ethyl acetate (100 mL). The organic phase was separated and was washed with water twice, concentrated and dried by Na₂SO₄ to obtain a crude product; the crude product was purified by silica gel column chromatography (dichloromethane:methanol=30:1) to obtain a pure compound 1 (1.7 g, yield: 58%), a white powder.

¹HNMR(DMSO-d6): δ=11.41(s, 1H, NH), 7.83-7.71(d, 1H, CH), 5.84-5.78(d, 1H, CH), 5.65-5.61(d, 1H, CH), 4.26-4.18(m, 1H, CH), 4.10-4.03(m, 1H, CH), 3.97-3.91(m, 1H, CH₂), 3.90-3.82(m, 1H, CH), 3.75-3.67(m, 1H, CH₂), 0.95-0.87(m, 18H, CH₃), 0.86-0.78(m, 9H, CH₃), 0.14-0.07(m, 18H, CH₃).

ESIm/z calcd for C₂₇H₅₄N₂O₆Si₃, exact mass: 586.33, and found [M+H⁺]: 587.34.

### EXAMPLE 2

### Synthesis of 4-(aminooxy)-1-(1R,2R,3R,4R,5R)-3,4-bis((tert-butyldimethylsilyl chloride(yl))oxy)-5-(((tert-butyldimethylsilyl chloride(yl)oxy)methyl)tetrahydrofuran pyridine-2-yl)pyrimidin-2(1H)-one)

The title compound was synthesized via the following reaction:

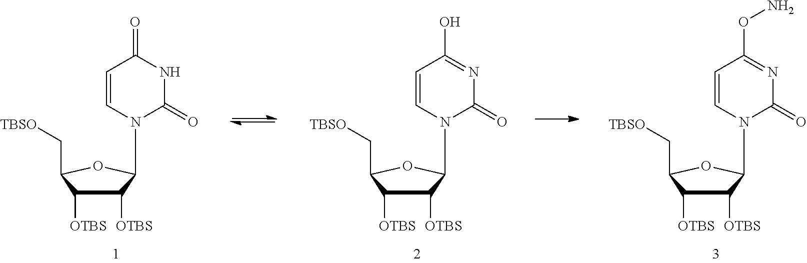

The compound 1 (Example 1) (880 mg, 1.5 mmol) was dissolved in 4 mL methanol, and then added with potassium tert-butoxide (168 mg, 1.5 mmol); the reaction mixture was stirred for 0.5 h under nitrogen protection. Methanol was evaporated at low temperature and the residual was dissolved in 4 mL dichloromethane. The reactor was placed on an ice-water mixture, and a dichloromethane (4 ml) solution of O-(mesitylene sulfonyl) hydroxylamine (MSH) (427 mg, 1.5 mmol) was added. The above obtained solution was stirred overnight and centrifuged to separate the liquid. The liquid was concentrated and purified by silica gel column chromatography (dichloromethane:methanol=30:1) to obtain a white crystalline compound 3 (600 mg, yield: 68%).

¹H NMR(DMSO-d6): δ=7.85-7.81(d, 1H, CH), 5.82-5.80(m, 1H, CH), 5.80-5.78(d, 1H, CH), 5.53-5.46(br, 2H, NH₂), 4.28-4.24(m, 1H, CH), 4.10-4.07(m, 1H, CH), 3.99-3.95(m, 1H, CH₂), 3.94-3.89(m, 1H, CH), 3.75-3.71(m, 1H, CH₂), 0.91-0.84(m, 27H, CH₃), and 0.10--0.04(m, 18H, CH₃).

ESIm/z calcd for C₂₇H₅₅N₃O₆Si₃, exact mass: 601.34, and found [M+H⁺]: 602.35.

### EXAMPLE 3

### Synthesis of 4-(aminooxy)-1-((2R,3R,4S,5R)-3,4-dihydroxy-5-(hydroxymethyl)tetrahydrofuran-2-yl)pyrimidine-2(1H)-one(4-aminooxycytidine)

The title compound was synthesized via the following reaction:

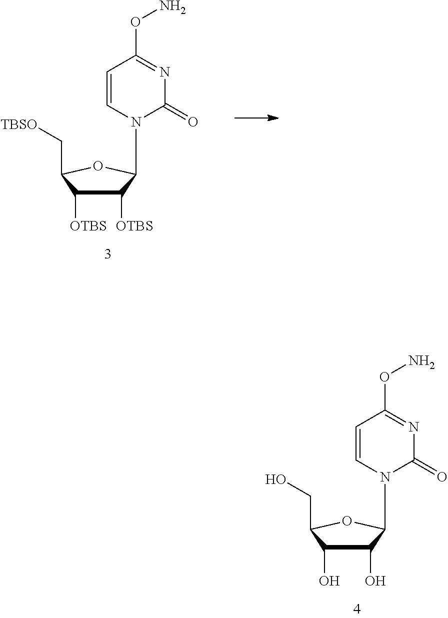

1 M tetrabutylammonium fluoride in a tetrahydrofuran solution (0.7 mL, 0.7 mmol) was added to an anhydrous tetrahydrofuran solution of the compound 3 (120 mg, 0.2 mmol). The reaction mixture was stirred for 4 h at room temperature. The solvent was removed. 1 mL methanol and a drop of ammonia water were added to the reaction mixture to alkalize the reaction. The reaction mixture was purified using silica gel column chromatography (ethyl acetate:ethanol=4:2) to obtain a white solid compound 4 (20 mg, yield: 39%).

¹H NMR(DMSO-d6): δ=7.99-7.91(d, 1H, CH), 5.86-5.81(m, 2H, CH), 5.51-5.48(br, 2H, NH2), 5.45-5.42(d, 1H, OH), 5.15-5.10(m, 2H, OH), 4.08-4.03(m, 1H, CH), 4.00-3.96(m, 1H, CH), 3.90-3.86(m, 1H, CH₂), 3.68-3.62(m, 1H, CH), and 3.60-3.54(m, 1H, CH₂). ESIm/z calcd for C₉H₁₃N₃O₆, exact mass: 259.08, and found [M+N⁺] 282.02.

### EXAMPLE 4

### Materials and Methods

The NMR spectroscopy was measured using a Bruker 400 MHz NMR spectrometer. Mass spectrum (ESI) was measured using a Thermo q-exactive mass spectrometer. Thin layer chromatography was generated using a Merck TLC Silica Gel 60 F2541 fluorescence analysis plate. Column chromatography was generated using silica gel with the specification of 200 to 300 meshes. The reactions were under the protection of N₂. All reagents were purchased from Sigma-Aldrich and SCRC, and used without further purification. Reaction solvents were anhydrous reagents.

### EXAMPLE 5

### Synthesis of 4-aminooxycytidine-5′-triphosphate or 4-aminooxydeoxycytidine-5′-triphosphate The 4-aminooxycytidine-5′-triphosphate or 4-aminooxydeoxycytidine-5′-triphosphate Disclosed herein Might be Synthesized via the Following Reaction

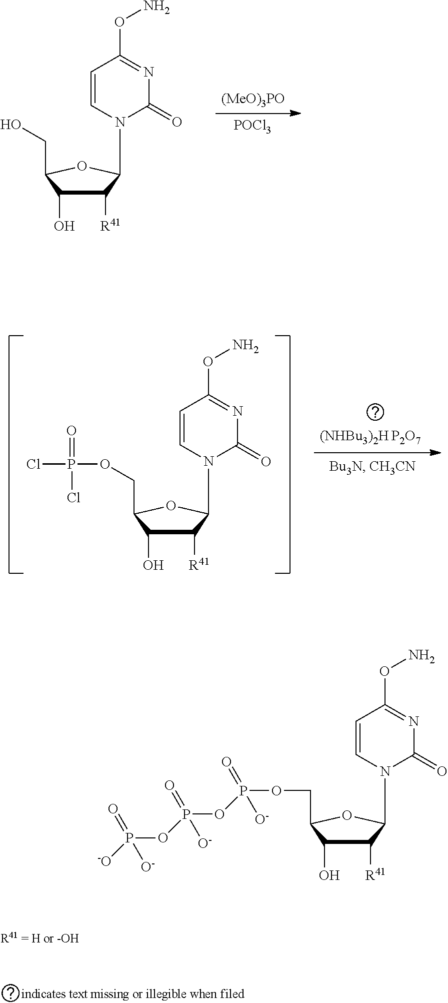

To a stirred solution of 4-aminooxycytidine (R⁴¹=—OH) or 4-aminooxydeoxycytidine (R⁴¹=H) (3.89 mmol) in trimethyl phosphate (20 mL) at 0° C., phosphorous oxychloride (0.36 mL, 3.87 mmol) was added and the mixture was stirred for 10 mins. Another portion of phosphorous oxychloride (0.36 mL, 3.87 mmol) was added to the reaction mixture and was further stirred for 40 mins. A pre-cooled mixture containing tributylammonium pyrophosphate (5.29 g, 9.67 mmol), tributylamine (5.60 mL, 23.49 mmol) and acetonitrile (15 mL) was added to the reaction mass and kept under stirring for 10 mins. The reaction mixture was quenched by slow addition of 500 mL water followed by extraction with dichloromethane (3×100 mL). The collected aqueous solution was adjusted to pH 6.5 and loaded on a DEAE Sepharose column. The desired product was eluted using a linear gradient of 0-1 M TEAB and the fractions containing the product were pooled, evaporated, and co-evaporated with water (3×100 mL). The obtained TEA salt was subjected to ion-exchange with sodium perchlorate (5.0 g) in acetone (100.0 mL) for two times to obtain the sodium salt of 4-aminooxycytidine-5′-triphosphate or 4-am inooxydeoxycytidine-5′-triphosphate.

### EXAMPLE 6

### Synthesis of O-(formyl sulfonyl) hydroxylamine (MSH)

MSH disclosed in Example 2 was synthesized by the following reaction:

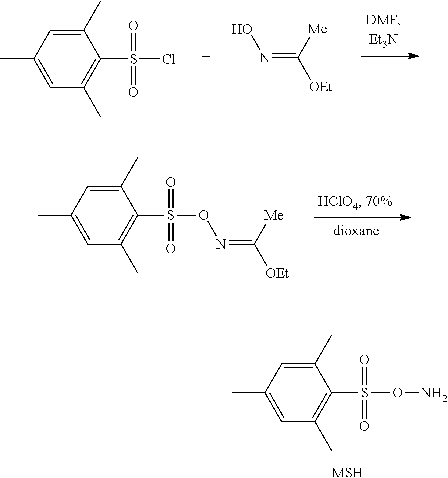

O-(mesitylene sulfonyl)ethyl acetohydroxamate (7.5 g) were dissolved into dioxane (5 ml); stirred and cooled to 0° C. 70% perchloric acid (3 ml) was dropwise added to the above solution to keep a temperature lower than 10° C. The obtained mixture was added to ice water (300 ml) to filter out crude MSH; the crude MSH was fully washed with water and dissolved in diethyl ether (30 ml). The ether solution was washed by water (25 ml) and treated with anhydrous potassium carbonate (5 g) for 30 s and then filtered. The ether solution was poured to cold pentane (300 ml) such that MSH was precipitated into small crystals; then the crystals were collected and vacuum dried at room temperature for 5 mins. Requirements as well as methods and structures within the range of equivalents thereof.

### Example 7

### Experiment on the Expression of a Modified mRNA of the Luciferace Reporter on Dendritic Cells

1.1 The mRNA of the luciferase reporter (FLuc) has the following sequence (Fluc mRNA, source: Trilink Biotechnologies) (natural):

(SEQ NO: 1)
5-AUGGAGGACG CCAAGAACAU CAAGAAGGGC
CCCGCCCCCU UCUACCCCCU
GGAGGACGGC ACCGCCGGCG AGCAGCUGCA
CAAGGCCAUG AAGCGGUACG CCCUGGUGCC
CGGCACCAUC GCCUUCACCG ACGCCCACAU
CGAGGUCCAC AUCACCUACG CCGAGUACUU
CGAGAUGAGC GUGCGGCUGG CCGAGGCCAU
GAAGCGGUAC GGCCUGAACA CCAACCACCG
GAUCGUGGUG UGCAGCGAGA ACAGCCUGCA
GUUCUUCAUG CCCGUGCUGG GCGCCCUGCC
CAUCGGCGUG GCCGUGGCCC CCGCCAACGA
CAUCUACAAC GAGCGGGAGC UGCUGAACAG
CAUGGGCAUC AGCCAGCCCA CCGUGGUGUU
CGUGAGCAAG AAGGGCCUGC AGAAGAUCCU
GAACGUGCAG AAGAAGCUGC CCAUCAUCCA
GAAGAUCAUC AUCAUGGACA GCAAGACCGA
CUACCAGGGC UUCCAGAGCA UGUACACCUU
CGUGACCAGC CACCUGCCCC CCGGCUUCAA
CGAGUACGAC UUCGUGCCCG AGAGCUUCGA
CCGGGACAAG ACCAUCGCCC UGAUCAUGAA
CAGCAGCGGC AGCACCGGCC UGCCCAAGGG
CGUGGCCCUG CCCCACCGGA CCGCCUGCGU
GCGGUUCAGC CACGCCCGGG ACCCCAUCUU
CGGCAACCAG AUCAUCCCCG ACACCGCCAU
CCUGAGCGUG GUGCCCUUCC ACCACGGCUU
CGGCAUGUUC ACCACCCUGG GCUACCUGAC
CUGCGGCUUC CGGGUGGUGC UGAUGUACCG
GUUCGAGGAG GAGCUGUUCC UGCGGAGCCU
GCAGGACUAC AAGAUCCAGA GCGCCCUGCU
GGUGCCCACC CUGUUCAGCU UCUUCGCCAA
GAGCACCCUG AUCGACAAGU ACGACCUGAG
CAACCUGCAC GAGAUCGCCA GCGGCGGCGC
CCCCCUGAGC AAGGAGGUGG GCGAGGCCGU
GGCCAAGCGG UUCCACCUGC CCGGCAUCCG
GCAGGGCUAC GGCCUGACCG AGACCACCAG
CGCCAUCCUG AUCACCCCCG AGGGCGACGA
CAAGCCCGGC GCCGUGGGCA AGGUGGUGCC
CUUCUUCGAG GCCAAGGUGG UGGACCUGGA
CACCGGCAAG ACCCUGGGCG UGAACCAGCG
GCGCGAGCUG UGCGUGCGGG GCCCCAUGAU
CAUGAGCGGC UACGUGAACA ACCCCGAGGC
CACCAACGGC CUGAUCGACA AGGACGGCUG
GCUGCACAGC GGCGACAUCG CCUACUGGGA
CGAGGACGAG GACUUCUUCA UCGUGGACCG
CCUGCUGCAG CACCCCAACA UCUUCGACGC
CGGCGUGGCC GGCCUGCCCG ACGACGACGC
CCGCGAGCUG CCCGCCGCCG UGGUGGUGCU
GCAGCACGGC AAGACCAUGA CCGAGAAGGA
GAUCGUGGAC UACGUGGCCA GCCAGGUGAC
CACCGCCAAG AAGCUGCGGG GCGGCGUGGU
GUUCGUGGAC GAGGUGCCCA AGGGCCUGAC
CGGCAAGCUG GACGCCCGGA AGAUCCGGGA
GAUCCUGAUC AAGGCCAAGA AGGGCGGCAA
GAUCGCCGUG UGA-3′

Obtaining of the modified luciferase mRNA: DNA sequence of luciferase may be in vitro transcribed into mRNA using a transcriptase under a conventional reagent condition. During the process of transcription, modified mRNAs with various modification rates were obtained according to a ratio of the modified C (cytidine) and unmodified C, wherein the modified mRNA may contain U-modified mRNA in varying ratios. The sequence was in vitro synthesized into the following modified mRMA, thus forming new modified luciferase. In the above sequence (SEQ NO:1), cytidine was replaced by the modified C* according to the present disclosure; and the following modified C* denotes the expression of the mRNA of 5 monocytidine-modified mRNA having a modification rate of 100% (namely, all the C were replaced with the following 5 different C* modifications, m⁴C (N4-methylcytidine), and m⁴Cm (N⁴, 2′-O-dimethylcytidine), specifically as shown in Table 1.

It can be seen that there are a variety of methods or ways to synthesize the modified mRNA; and any existing method for synthesizing the modified mRNA can be applied in the present disclosure for implementation. A commercial kit may be purchased for in vitro transcription. Such kind of implementation may achieve 100% modification, or a certain rate of modification, such as, 90%, 85%, 80%, 75%, 60%, 50%, 40%, 20%, 10%, 2% or 0.5% modification. For example, all the cytidine on the above-mentioned FLuc mRMA may be substituted into modified cytidine, for example, substitution by any chemical structure according to the present disclosure, and also for example, substitution by the specifically modified cytidines of the compounds 1, 2, 3 and 4 illustrated hereafter; and the substitution ratio may be 100%, and also different certainly. Such kind of substitution also may be mixed replacement with various forms of modification instead of being a single replacement. For example, for the modification of cytidine, the cytidine in certain positions may be replaced by one or more of the specific compounds of 1, 2, 3, and 4. Such kind of method for producing modified mRNA is, for example, specifically described in a Chinese invention patent CN102947450B; and each method in the description of the patent is used through a specific example of the present disclosure.

TABLE 1
Number of experimental treatments in Example 1

| Control | Invention 1 | Invention 2 | Invention 3 | m4C | m4Cm | Invention 4 |
|---|---|---|---|---|---|---|
| 0% | 100% | 100% | 100% | 100% | 100% | 100% |

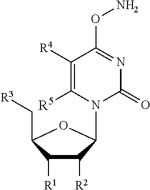

(Invention 1, wherein R4 is H, R5 is H, R2 is —OH, R1 is —OH, R3:
namely, R3 is —OH, wherein the H in —OH is substituted by
triphosphoryl, namely, —O-potassium triphosphate). 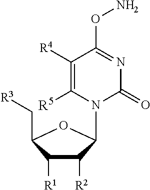
(Invention 2, wherein R4 is —CH3, R5 is —H, R2 is —H, R1 is —OH,
R3 is —OH, wherein the H substituted by triphosphate, namely,
—O-potassium triphosphate). 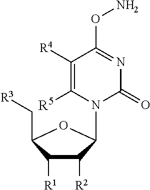
(Invention 3, wherein R4 is —OH, R5 is —NH2, R2 is —OH, R1 is —OH,
R3: namely, R3 is —CH2—OH, wherein the H is substituted by triphosphoryl,
namely, —O-potassium triphosphate). 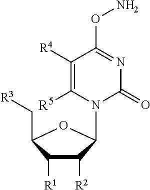
(Invention 4, wherein R4 is —OH, R5 is —CH3, R2 is —OH, R1 is —OH,
R3: namely, R3 is —OH, and the H is substituted by triphosphoryl, namely,
—O-potassium triphosphate).

1.2 LPP encapsulation process was performed according to the following method:

1.2.1: Preparation of a phospholipid mixed liquor:phospholipid:DOPE:mPEG2000-DSPE=49:49:2 was dissolved in an ethanol solution in proportion, wherein DOPE was purchased from Avanti, mPEG2000-DSPE was purchased from cordenpharma, and PBS was purchased from Invitrogen.

1.2.2: Preparation of mRNA: 1 mL each of the treated mRNAs (mRNA having a concentration of 0.2 mg/mL and total mass of 0.2 mg as shown in Table 2) was respectively sucked up by a BD injector.

1.2.3: Preparation of phospholipid/mRNA: 3 mL mRNA and 3 mL phospholipid solution (having a concentration of 12 mg/mL) were respectively sucked up by a BD injector and inserted into a micro-fluidic chip (the microfluidics herein should be a small-sized equipment capable of producing package for nanoparticles); the setup parameters: volume: 9.0 mL; and flow rate ratio: 3:1, total flow rate: 1 mL/min; temperature: 37.0° C., initial amount: 0.35 mL; ending amount: 0.10 mL, thus a phospholipid/mRNA solution was obtained, namely, mRNA particles encapsulated by phospholipid and a phospholipid mixed solution were obtained.

1.2.4: Centrifugal ultrafiltration: the phospholipid/mRNA solution was added to an ultrafiltration tube for centrifugal ultrafiltration, the sample volume was 12 mL, and the ultrafiltration medium, a phosphate buffer had a volume of 12 mL; and ultrafiltration parameters were set as follows: centrifugal force was 3400 g, centrifugation time was 60 min, temperature was 4° C., and number of cycles was 3. Thereby, each of the treated encapsulated mRNA carriers was obtained.

Encapsulation method in the detailed example is a LPP method; any other methods can be certainly used to encapsulate the mRNA, or naked mRNA without encapsulation is directly used to transfect cells, tissues, any viable tissues, or the like. Certainly, a gene gun or a transgenic method may be used to transfer mRNA to cells for the expression of a target protein. These are conventional methods in the prior art.

1.3 Cell transfection experiment

Experimental reagents: (1) water was added to a harvest buffer (25 ml): 1.25 ml 1 M Tris-HCl (pH7.5), 25 μl 1 M DTT, 250 μl 10% Triton X-100 to 25 ml for storage at 4° C. (2) Water was added to an ATP buffer (10 ml): 1.25 ml 1 M Tris-HCl (pH7.5), 250 μl 1 M MgCl2, 24 mg ATP to 10 ml for storage at 20° C. (3) Luciferin buffer (36 ml): 10 mg luciferin, 36 ml 5 mM KH2PO4 (pH7.8) was performed for storage at 4° C. (4) PBS: 20 mMNaCl, 2.68 mMKCl, 10 mM Na2HPO4, 1.76 mM KH2PO4 was performed for storage at 4° C.

Certainly, a commercial kit can be used to test the expression quantity of luciferase, and the amount of the expression quantity may directly indicate the expression quantity of mRNA.

Each of the treated carriers encapsulated with mRNA obtained in 1.2 was used to transfect dendritic cells, and specifically as follows (3 times were repeated for each treatment):

1.3.1: mice dendritic cells (purchased from FENGHUISHENGWU, D.2.4 cells) were digested and seeded in a 35 mm petri dish on the first day of the experiment, then placed in a 37° C. incubator (5% CO2, saturated humidity) for overnight culture;

1.3.2: cells were transfected by each treatment when cell density was up to 70%;

1.3.3: culture medium was sucked away 24 h after transfection, and cells were washed by ice-cold PBS.

Note: the enzymatic reaction of luciferase will be inhibited by trace calcium; therefore, cells transfected by calcium phosphate should be thoroughly washed to remove the calcium-containing medium before collection.

1.3.4: 350 μl precooled harvest buffer was added to each petri dish, and then put at 4° C. or on ice for 10 min for cell lysis.

1.3.5: during cell lysis, plenty of 1.5 mL microcentrifuge tubes were prepared, ATP buffer and luciferin buffer were mixed at a ratio of 1:3.6 to form a reaction solution, then 100 μl reaction solution was aliquoted into each tube.

1.3.6: equal volume of cell lysis buffer (100 μl) was successively taken and added into the microcentrifuge tubes in step 5, and mixed evenly immediately, and absorbance values were read on a Luminometer. Note: the luminous reaction will attenuate rapidly; the absorbance value must be read within 5 s after adding the cell lysis buffer to the reaction solution.

1.3.7: ensured to read the absorbance values of all the samples by the same operating method.

1.3.8: the remaining lysate was taken to measure the activity of LacZ, and the reading served as an internal standard to correct the reading of the luciferase.

1.3.9: the corrected reading was used for plotting and data analysis (see FIG. 2). Note: luciferin is easy to be oxidized when it is exposed to the light, and the luciferin which has been diluted but not used should be discarded.

1.4 Result analysis

It can be seen from FIG. 2 that compared with the control (cytidine without modification), the content of the expressed protein (luciferase) in cells has an obvious change with the change of the various modification forms of cytidine. The expression quantities in cells from all of the three modification forms of cytidine according to the present disclosure were higher than that from the control. Through the significance analysis on variance, Inventions 1, 2, 3, and 4 respectively showed a highly significant difference (P<0.01) compared with the control, m4C and M5U.

It indicates that the use of the modified cytidine according to the present disclosure has remarkable improvement or influence on the expression of mRNA. The influence may improve the stability of mRNA in the encapsulation carrier, and meanwhile may also influence that after encapsulated in the carrier, the carrier is transported into cells, the mRNA has a higher stability and better translation properties, and is expressed into proteins with higher stability and activity. These multiple aspects of influences will finally determine the activity or quantity of the luciferase. Thereby, the luciferase has a superiority over the unmodified control samples (FIG. 2).

To make (mRNA) having higher stability, some structures may be added on the 5′ terminal or 3′ terminal of a messenger RNA exerting core functions, such that it has a higher stability and protein translation ability. Such kind of additional structure may be achieved easily in the prior art. For example, to prevent mRNA degradation and enhance the stability, it usually needs to add a proper tail on the 3′ terminal of mRNA. Therefore, accurately reflecting the Poly(A) tail length is very important to the quality control of mRNA production process. Majority of the eucaryon are provided with Poly(A) tails, namely, mRNA Poly(A) tails, consisting of 100-200 A on the 3′ terminal of mRNA. The mRNA Poly(A) tail is not encoded by DNA, but catalytic polymerized onto the 3′ terminal of transcribed premessenger RNA by an RNA-terminal adenine nucleotide translocase with ATP as a precursor. The known functions of the mRNA Poly(A) tail are: \textcircled{1} facilitating the transport of mRNA to cytoplasm from the cell nucleus; \textcircled{2} avoiding degradation in cells by ribozyme and enhancing the mRNA stability; \textcircled{3} serving as an identification signal for the ribosome. Such kind of structure for Poly(A) addition also may be achieved in vitro.

It is also known in the art that mRNA molecules usually have regions of different sequences located before the translation initiation codon and after the untranslated translation termination condon. These regions (respectively called a 5′ untranslated region (5′UTR) and a 3′ untranslated region (3′UTR)) may influence the mRNA stability, mRNA positioning and the translation efficiency of the mRNA linked therewith. It is known that some 5′ and 3′ UTR, for example, 5′ and 3′ UTR of α- and β-globins may improve the mRNA stability and mRNA expression. Therefore, in some preferred embodiments, mRNA encoding reprogramming factors (e.g., an iPSC inducing factor) exhibited 5′ UTR and/or 3′ UTR (for example, the 5′ UTR and/or 3′ UTR of α-globin or β-globin; for example, the 5′ UTR and/or 3′ UTR of Xenopus laevis or human α-globin or β-globin; or for example, the 5′UTR of tobacco etch virus (TEV)) resulting in higher mRNA stability and higher mRNA expression in cells.

Specifically, the (mRNA) with core functions has a higher stability and superiority of other inventions, which may be achieved by the technical solution disclosed in the following patent application. For example, the method described in the description of a Chinese invention patent CN102947450B serves as a portion of the present disclosure.

### EXAMPLE 8

### Influence of In Vitro Tailing Structure (Poly (A) of mRNA on the Expression of the Modified mRNA (Dendritic Cells)

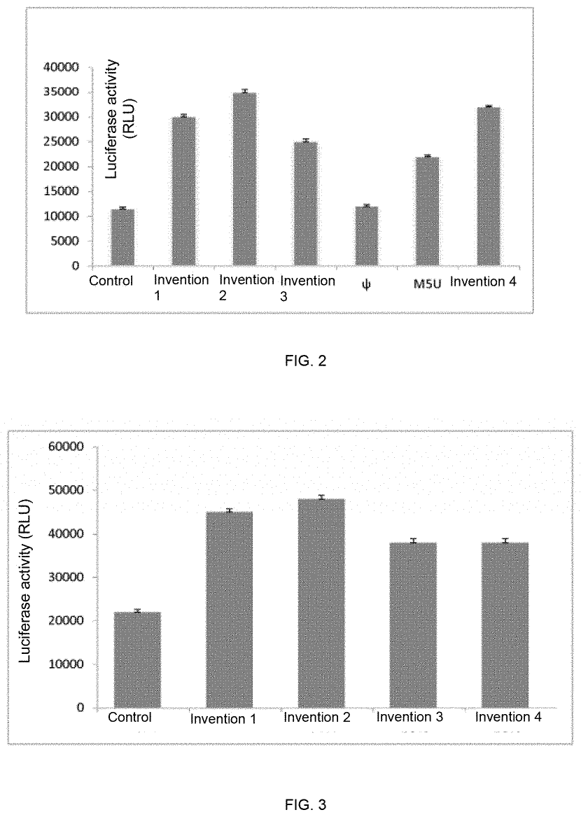

120 A was added on the 3′ terminal as a tail, so as to investigate the influence of adding Poly(A) on the 3′ terminal on the translation effect, as well as on the expression of the luciferase 100% modified by Inventions 1-4 and the control. The influence on enzyme expression was measured according to the encapsulation method and cell transfection method in Example 1. It can be obviously seen through the comparison of FIG. 3 that the expression was increased both for the modified or unmodified mRNA after adding a Poly (A) structure on the 3′ terminal. However, there were some differences among the various modification ways; the extents of increase for Inventions 1-4 comparing to the control are different.

In a similar experiment HEK293 cells were transfected, the results were different, and the overall expression level was 2-3 folds higher than that in dendritic cells, but the trend was similar (specific data were omitted).

### EXAMPLE 9

### Influence of Various Modification Rates on mRNA Expression

mRNA of the luciferase in Example 1 was set as an example; a portion of cytidine was replaced with the compounds of the modified cytidine according to the present disclosure with a rate of 0.5%, 5%, 10%, 20%, 30%, 40%, 50%, 70%, 80% and 90%; and the specific replacement method was as follows: in vitro transcription of the luciferase DNA was performed by a transcriptase with the supply of AUCG as raw materials according to a conventional method, wherein the synthesis method should be controlled and replaced, and then a portion of cytidine in mRNA was replaced according to the above rate. The influence of various replacement rates on the expression of mRNA was investigated. The specific investigation was performed by reference to the method of Example 1, and the results are shown in the figure below.

It can be seen from FIG. 4 that for the replacement of the unmodified cytidine with the modified cytidine of Invention 1, with the increase of the modification rate, the expression of the target mRNA also increases. There is a significant difference in the expression quantity between the modification rate of 5%-50% and other modification rates. It indicates that if a cytidine having the structure of Invention 1 is desired for mRNA modification, the modification ratio is greater than 5%.

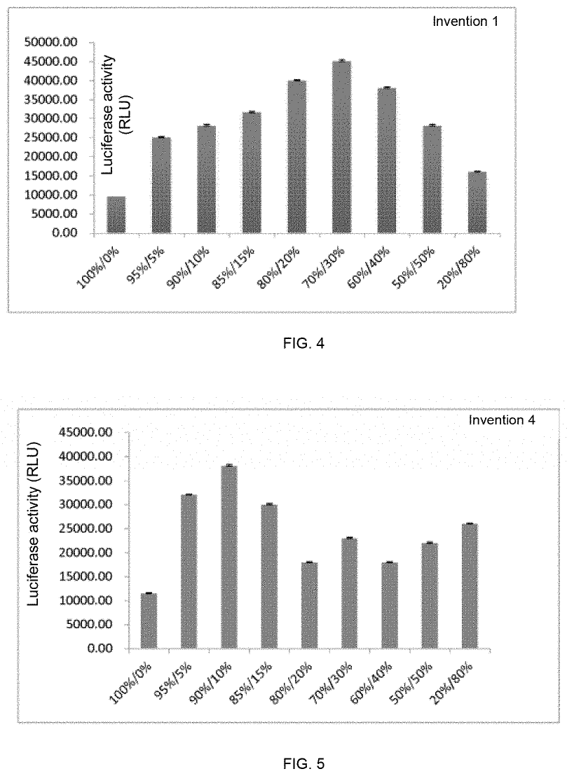

It can be seen from FIG. 5 that for the replacement of the unmodified cytidine with the cytidine of Invention 4, with the increase of the rate, the expression level also gradually increases, and when the modification rate is 5%-80%, the expression quantity is relatively high, and being the maximum when the rate is 10%. Through variance analysis, there is a significant difference or highly significant difference between the modification rate of 10% and other modification (the specific analysis process was omitted). It indicates that if cytidine having the structure of Invention 4 is desired for mRNA modification, the expression level is the maximum when the modification rate is about 10% .

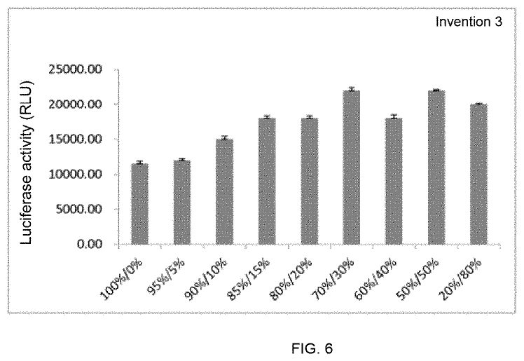

It can be seen from FIG. 6 that for the replacement of the unmodified cytidine with the modified cytidine of Invention 3, with the increase of the rate, the expression level also gradually increases, and when the modification rate is 10-80%, the expression quantity is relatively high, and being the maximum when the rate is 30%. Through variance analysis, there is a significant difference or highly significant difference between the modification rate of 30% and other modification (the specific analysis process was omitted). It indicates that if cytidine having the structure of Invention 2 is desired for mRNA modification, the expression level is up to the maximum when the modification rate is about 30%.

In addition, in these four different modification solutions with various modification rates according to the present disclosure, the maximum expression quantity is determined by an optimal modification rate. It may be that the difference of the substituents in different positions influences the final expression quantity, but it is probably because the 4-position of the common cytidine has a common structure change.

For the protein which is expected to achieve a high expression quantity in vivo, the in vivo expression quantity of the protein may be significantly improved by the way of replacing the cytidine in mRNA with the cytidine modification according to the present disclosure. The specific embodiments according to the present disclosure are subjected to experimental verification directed to luciferase, but it should be understood that for other mRNAs, for example, mRNAs for the treatment of certain cancers, mRNAs for infectious disease vaccines or therapeutic vaccines, or any other mRNAs, a proper rate may be found through the cytidine modification according to the present disclosure and reasonable experiments, thus significantly improving the expression quantity of the target mRNA in vivo. It is readily understood by a person skilled in the art; luciferase is a reporter gene for expression, and the increase of its expression quantity also indicates the increase of the target mRNA.

A person skilled in the art should understand that in this example, a conventional luciferase is merely used to verify that the compound according to the present disclosure may be used for replacing cytidine and achieving the modification effect. It is described only by way of illustration, but cannot be regarded to have effect on luciferase only. On the contrary, luciferase is merely a conventional tool for verification, and definitely may be used for those meaningful nucleic acids, for example, modification of messenger RNAs, the mRNAs of genes associated with lots of cancers or tumors, mRNAs of infectious diseases, or any other related mRNA modification, which also has effects and functions. Certainly, it also includes the mRNA modification related to any plant, animal, bacteria and algae; mRNA may be modified by the modified cytidine compound according to the present disclosure to significantly improve the expression and translation of the target mRNA in cells.

All the patents and publications mentioned in the present disclosure represent the technical disclosures in the art, and can be used herein. All the patents and publications cited therein are similarly listed in the references, and are the same as the specific citing alone of each publication. The present disclosure herein may be implemented in case of lacking any one or more elements, one or more limitations; and the limitation is not stated specifically herein. For example, terms “comprise”, “substantively consisting of . . . ” and “consisting of . . . ” in each example herein may be replaced by the rest two terms. The so-called “a/an” herein merely denotes the meaning of “one”, which does not exclude including only one, and also denotes including two or more. Terms and expressions used herein are ways of description, and are not limited thereto; neither is there any intention to indicate that these terms and explanations described herein exclude any equivalent features. However, it is understood that any suitable changes or modifications may be made within the scope of the disclosure and the claims. It should be understood that examples described herein are preferred examples and features. Any person skilled in the art may make some alterations and changes within the spirit of the present disclosure. These alterations and changes are deemed to be within the scope of the present disclosure as well as the scope defined by the independent and dependent claims.

1. A compound of Formula (I): 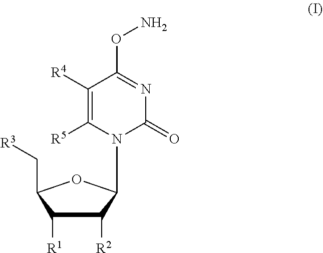
or a pharmaceutically acceptable salt thereof, wherein:
R¹, R², R⁴, and R⁵ are each independently selected from the group consisting of —H, —OH, —NH₂, halo, substituted or unsubstituted C₁-C₁₀ alkyl, substituted or unsubstituted aryl, substituted or unsubstituted heteroaryl, substituted or unsubstituted C₁-C₁₀ aralkyl, substituted or unsubstituted C₁-C₁₀ cycloalkyl, substituted or unsubstituted C₁-C₁₀ heterocyclyl, substituted or unsubstituted acyl, —OR⁶, —C(O)R⁶, —C(O)—O—R⁶, —C(O)—NH—R⁶, and —N(R⁶)₂;
R³ is selected from the group consisting of —H, —OH, —NH₂, halo, substituted or unsubstituted C₁-C₁₀ alkyl, substituted or unsubstituted aryl, substituted or unsubstituted heteroaryl, substituted or unsubstituted C₁-C₁₀ aralkyl, substituted or unsubstituted C₁-C₁₀ cycloalkyl, substituted or unsubstituted C₁-C₁₀ heterocyclyl, substituted or unsubstituted acyl, —OR⁶, —C(O)R⁶, —C(O)—O—R⁶, —C(O)—NH—R⁶, and —N(R⁶)₂, phosphate, diphosphate, and triphosphate; and wherein R⁶ is —H, substituted or unsubstituted C₁-C₁₀ alkyl, or substituted or unsubstituted acyl.

2. The compound of claim 1, wherein R¹, R², R⁴, and R⁵ are each independently —H, —OH, or substituted or unsubstituted C₁-C₁₀ alkyl.

3. The compound of claim 1, wherein R³ is —H, —OH, substituted or unsubstituted C₁-C₁₀ alkyl, phosphate, diphosphate, or triphosphate.

4.-8. (canceled)

9. The compound of claim 41, having the structure of Formula (I-a), Formula (I-b), Formula (I-c), Formula (I-d), Formula (I-e), or Formula (I-f):

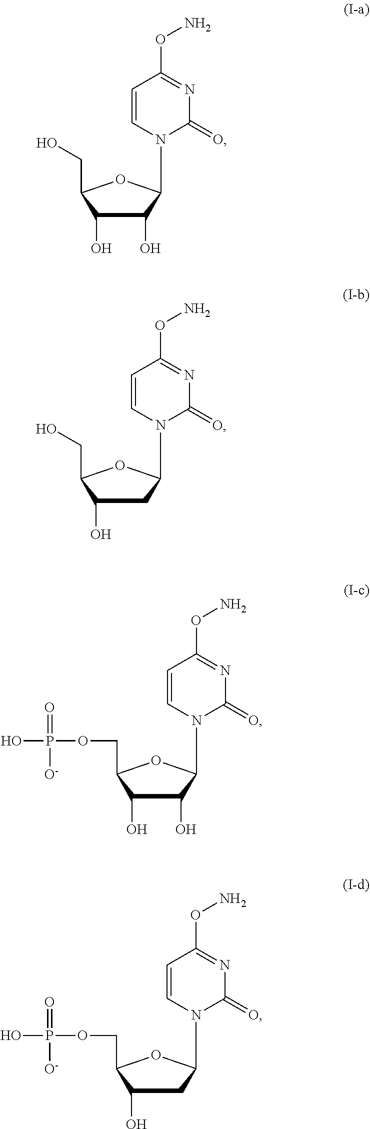

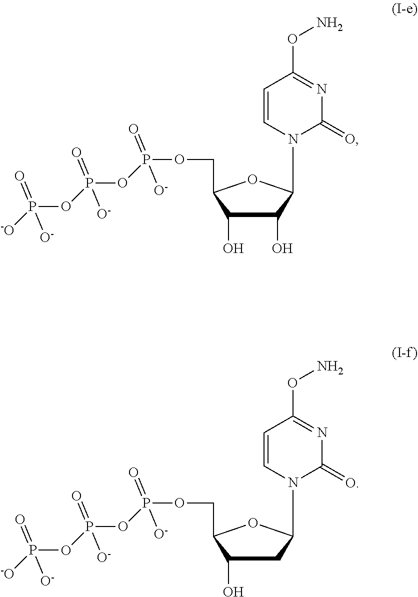

10.-19. (canceled)

20. A modified nucleoside triphosphate (NTP) having the structure of Formula (I-g) or Formula (I-h): 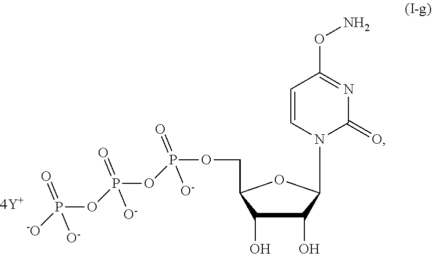
wherein, Y⁺ is a cation; 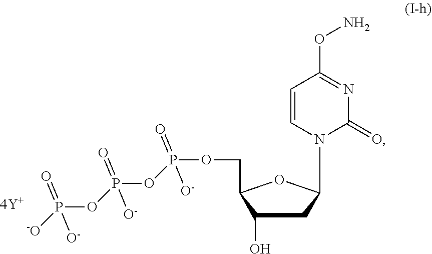
wherein, Y⁺ is a cation.

21. (canceled)

22. The modified nucleoside triphosphate of claim 20, wherein the Y⁺ is selected from the group consisting of Li⁺, Na⁺, K⁺, H⁺, NH₄⁺, and tetraalkylammonium ions.

23. The modified nucleoside triphosphate of claim 22, wherein the tetraalkylammonium is selected from the group consisting of tetraethylammonium, tetrapropylammonium, and tetrabutylammonium.

24.-27. (canceled)

28. A nucleic acid, comprising two or more covalently bonded nucleotides, wherein at least one of the two or more covalently bonded nucleotides comprises the compound of claim 1.

29. The nucleic acid of claim 28, wherein the nucleic acid is a ribonucleic acid (RNA).

30. The nucleic acid of claim 29, wherein the RNA comprises the compound of

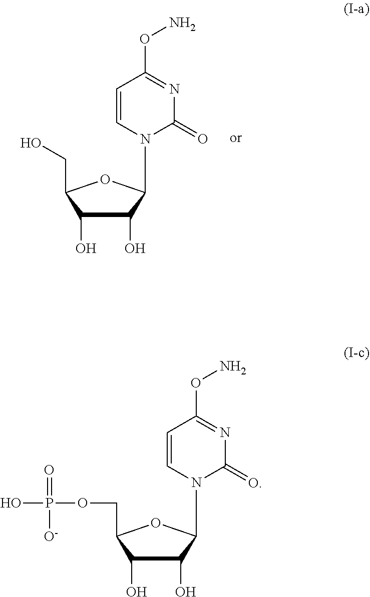

31. The nucleic acid of claim 29, wherein the RNA is a messenger RNA (mRNA).

32. The nucleic acid of claim 31, wherein the nucleotide comprises a modified cytidine of the following structure: 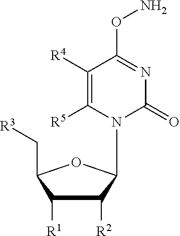
wherein, R⁴ is H; R⁵ is H; R² is —OH; R¹ is —OH; and R³ is —OH, wherein H is substituted by triphosphate;
or
wherein, R⁴ is —OH; R⁵ is —NH₂; R² is —OH; R¹ is —OH; and R³ is —CH₂—OH, wherein H is substituted by triphosphate;
or
wherein, R⁴ is —OH; R⁵ is —CH₃; R² is —OH; R¹ is —OH; and R³ is —OH, wherein H is substituted by triphosphate.

33. The nucleic acid of claim 32, wherein the modification rate is 30%-100%, 10-40%, 20-40%, or 70%-90%.

34.-41. (canceled)

42. A pharmaceutical composition, comprising the compound of claim 1, or a pharmaceutically acceptable salt thereof; and a pharmaceutically acceptable excipient.

43. A pharmaceutical composition, comprising the nucleic acid of claim 28; and a pharmaceutically acceptable excipient.

44. A compound of Formula (II): 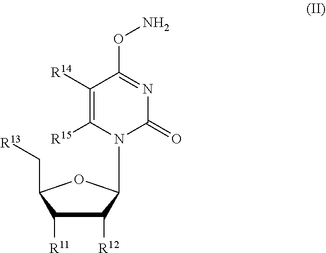
or a pharmaceutically acceptable salt thereof, wherein:
R¹¹, R¹², and R¹³ are each independently —H, —OH or —O— protecting group; R¹⁴ and R¹⁵ are each independently selected from —H, substituted or unsubstituted C₁-C₁₀ alkyl, or substituted or unsubstituted acyl.

45. The compound of claim 44, wherein R¹¹, R¹² and R¹³ are a —O— protecting group.

46. The compound of claim 45, wherein the protecting group is selected from the group consisting of acetyl, benzoyl, benzyl, β-methoxyethoxymethyl ethers, dimethoxytrityl [bis-(4-methoxyphenyl)phenylmethyl], methoxymethyl ethers, methoxytrityl [(4-methoxyphenyl)diphenylmethyl], p-methoxybenzyl ethers, methylthiomethyl ethers, pivaloyl, tetrahydropyranyl, tetrahydrofuranyl, trityl (triphenylmethyl), silyl ethers, methyl ethers, and ethoxyethyl ethers.

47. The compound of claim 44, wherein the protecting group is a silyl ether selected from the group consisting of trimethylsilyl ether (TMS), tert-butyldiphenylsilyl ether (TBDPS), tert-butyldimethylsilyl ether (TBDMS), and triisopropylsilyl ether (TIPS).

48. (canceled)

49. The compound of claim 44, having the structure of: [chemical structure]
